(19) United States
(12) Patent Application Publication
(10) Pub. No.: US 2012/0234380 A1
(43) Pub. Date: Sep. 20, 2012

(54) TERRYLENE COMPOUNDS, PREPARATION THEREOF AND USE THEREOF IN ORGANIC SOLAR CELLS

(75) Inventors: Martin Könemann, Mannheim (DE); Gabriele Mattern, Schifferstadt (DE); Jae Hyung Hwang, Viernheim (DE); Regina Höh, Waldsee (DE); Christian Dörr, Niederkirchen (DE); Junichi Tanabe, Mannheim (DE)

(73) Assignee: BASF SE, Ludwigshafen (DE)

(21) Appl. No.: 13/418,989

(22) Filed: Mar. 13, 2012

Related U.S. Application Data
(60) Provisional application No. 61/452,202, filed on Mar. 14, 2011.

(51) Int. Cl. H01L 51/46; C07D 311/78; H01L 31/0725; C07D 221/18; B82Y 99/00

(52) U.S. Cl. 136/255; 136/263; 546/26; 549/381; 977/948

(57) ABSTRACT

A terrylene compound of formula I:

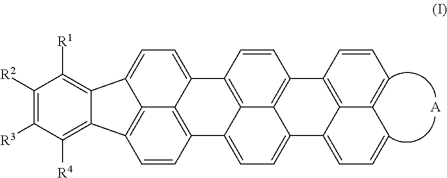

wherein R¹ to R⁴ are each independently an unsubstituted or substituted alkyl, aryl, thiophenyl or oligothiophenyl group, or a hydrogen atom. Two adjacent radicals selected from R¹ to R⁴, together with the carbon atoms of the benzene ring to which they are bonded, may also form a fused ring system having 1 to 8 additional rings. With the carbon atoms to which it is bonded, A forms a fused monocyclic, dicyclic, tricyclic, tetracyclic, pentacyclic or hexacyclic ring system which optionally contains one or more independently selected substituents. A process for preparing the terrylene compound. An organic solar cell with a photoactive region including at least one organic donor material in contact with at least one organic acceptor material, the donor material and the acceptor material forming a donor-acceptor heterojunction, and the photoactive region including at least one such terrylene compound.

### BACKGROUND OF THE INVENTION

The present invention relates to novel terrylene compounds and to processes for preparation thereof. The invention further relates to an organic solar cell with a photoactive region which comprises at least one organic donor material in contact with at least one organic acceptor material, wherein the donor material and the acceptor material form a donor-acceptor heterojunction and wherein the photoactive region comprises at least one such terrylene compound.

### STATE OF THE ART

Periflanthene derivatives, i.e. compounds with a diindeno[1,2,3-cd:1′,2′,3′-Im]perylene base skeleton:

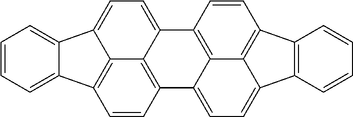

are the subject of intensive studies and have found use, for example, as chromophores and in systems based on electroluminescence, such as organic light-emitting diodes (OLEDs).

The synthesis and the electroluminescence of dibenzotetraphenylperiflanthene is described by J. D. Debad, J. C. Morris, V. Lynch, P. Magnus and A. J. Bard in J. Am. Chem. Soc. 1996, 118, p. 2374-2379.

M. Wehmeier, M. Wagner and K. Müllen describe, in Chem. Eur. J. 2001, 7, no. 10, p. 2197-2205, the preparation of novel perylene chromophores, including:

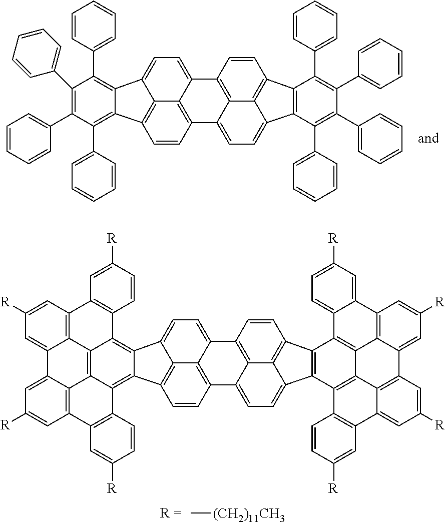

Owing to diminishing fossil raw materials and the CO₂ which is formed in the combustion of these raw materials and is active as a greenhouse gas, direct energy generation from sunlight is playing an increasing role. “Photovoltaics” is understood to mean the direct conversion of radiative energy, principally solar energy, to electrical energy.

In contrast to inorganic solar cells, the light does not directly generate free charge carriers in organic solar cells, but rather excitons are formed first, i.e. electrically neutral excited states in the form of electron-hole pairs. These excitons can be separated only by very high electrical fields or at suitable interfaces. In organic solar cells, sufficiently high fields are unavailable, and so all existing concepts for organic solar cells are based on exciton separation at photoactive interfaces (organic donor-acceptor interfaces or interfaces to an inorganic semiconductor). For this purpose, it is necessary that excitons which have been generated in the volume of the organic material can diffuse to this photoactive interface. The diffusion of excitons to the active interface thus plays a critical role in organic solar cells. In order to make a contribution to the photocurrent, the exciton diffusion length in a good organic solar cell must at least be in the order of magnitude of the typical penetration depth of light, in order that the predominant portion of the light can be utilized. The efficiency of an organic solar cell is characterized by its open-circuit voltage V_{OC}. Further important characteristics are the short-circuit current I_{SC}, the fill factor FF and the resulting efficiency ┐, and also the external quantum yield IPCE.

The first organic solar cell with an efficiency in the percent range was described by Tang et al. in 1986 (C W. Tang et al., Appl. Phys. Lett. 48, 183 (1986)). It consisted of a two-layer system with copper phthalocyanine (CuPc) as the p-semiconductor and perylene-3, 4:9, 10-tetracarboxylic acid bisimidazole (PTCBI) as the n-semiconductor. JP 2008-135540 describes the use of perylene derivatives of the general formula

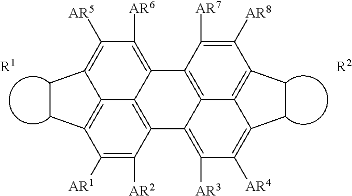

where R¹ and R² are each fused rings which may be substituted by alkyl, alkenyl, aryl, aralkyl or heterocyclyl, and AR¹-AR⁸ may each be alkyl, alkenyl, aryl, aralkyl or heterocyclyl, as an electron donor material for producing organic solar cells. In the examples, exclusively dibenzotetraphenylperiflanthene is used to produce an organic solar cell with photoactive donor-acceptor transitions in the form of a flat heterojunction. The use of differently substituted periflanthenes is not demonstrated, nor is the production of organic solar cells with donor-acceptor transitions in the form of a bulk heterojunction.

WO 2010/031833 describes the use of dibenzotetraphenylperiflanthene of the formula

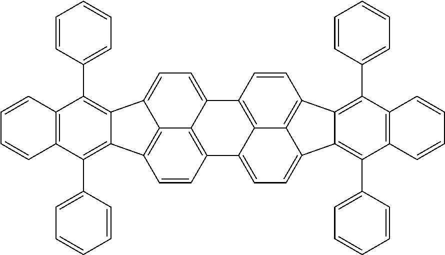

as an electron donor material in an organic solar cell with photoactive donor-acceptor transitions in the form of a bulk heterojunction.

WO 2011/000939 describes an organic solar cell with a photoactive region which comprises at least one substituted periflanthene of the formula (A)

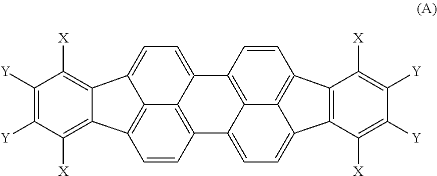

in which
X is independently selected from hydrogen and in each case unsubstituted or substituted alkyl, aryl, heteroaryl or oligo(het)aryl,
Y is independently selected from in each case unsubstituted or substituted alkyl, aryl, heteroaryl or oligo(het)aryl,
where in each case at least two adjacent radicals selected from the X and Y radicals, together with the carbon atoms of the benzene ring to which they are bonded, may also be a fused ring system having 1, 2, 3, 4, 5, 6, 7 or 8 further rings.

European patent application 10166498.5, which was yet to be published at the priority date of the present application, describes an organic solar cell with a photoactive region which comprises at least one substituted perylene of the formula (B)

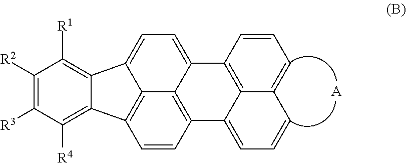

in which
R¹ and R⁴ are each independently selected from hydrogen, unsubstituted alkyl, substituted alkyl, unsubstituted aryl and substituted aryl,
R² and R³ are each independently selected from hydrogen, unsubstituted alkyl, substituted alkyl, unsubstituted aryl and substituted aryl, where in each case at least two adjacent radicals selected from the R¹, R², R³ and R⁴ radicals, together with the carbon atoms of the benzene ring to which they are bonded, may also be a fused ring system having 1, 2, 3, 4, 5, 6, 7 or 8 further rings, and
A with the carbon atoms to which it is bonded is a fused monocyclic, dicyclic, tricyclic, tetracyclic, pentacyclic or hexacyclic ring system with at least one exocyclic keto group, and where the ring system optionally bears one or more substituents bonded via a single bond.

It is a current aim in organic photovoltaics to provide a new generation of solar cells which are much less expensive than solar cells made from silicon or other inorganic semiconductors, such as cadmium indium selenide or cadmium telluride. For this purpose, there is still a need for suitable semiconductive, light-absorbing materials. One means of absorbing large amounts of light and of achieving good efficiencies is to use a pair of semiconductor materials which are complementary with regard to light absorption, for example comprising an n-semiconductor with short-wave absorption and a p-semiconductor with long-wave absorption. This concept also forms the basis of the abovementioned first organic solar cell, the so-called Tang cell. Even though many fullerene compounds absorb the light only weakly, it has been found that efficient solar cells can be produced when fullerenes or fullerene derivatives, such as C60 or C72, are used as n-semiconductors. It is additionally known, when using weakly absorbing semiconductor materials, that two solar cells can be built one on top of another. In that case, one cell comprises a combination of the weakly absorbing semiconductor with a complementary semiconductor which absorbs the short-wave radiation, and the other cell a combination of the weakly absorbing semiconductor with a complementary semiconductor which absorbs the long-wave radiation. For such tandem cells for combination with fullerenes or fullerene derivatives, two suitable p-semiconductors are required, one of which absorbs the short-wave radiation and one the long-wave radiation. The discovery of suitable semiconductor combinations is not trivial. In tandem cells, the open-circuit voltages Voc of the individual subcells are additive. The total current is limited by the subcell with the lowest short-circuit current Isc. The two semiconductor materials of the individual cells thus have to be matched exactly to one another. There is therefore a great need for semiconductive organic absorber materials with long-wave absorption for use in organic solar cells in combination with fullerenes or fullerene derivatives, and especially in tandem cells with high open-circuit voltage and acceptable short-circuit current.

It has now been found that, surprisingly, certain terrylene compounds are particularly advantageously suitable as semiconductive organic absorber materials. They are notable for their long-wave absorption and their suitability for combination with fullerenes and fullerene derivatives. They are especially suitable for use in the subcell of a tandem cell which absorbs the long-wave radiation.

For the person skilled in the art, especially the sublimeability of the terrylene compounds found is surprising. While the lower homologs, the perylenes, sublime more or less efficiently, this was not foreseeable for the compounds now discovered. For instance, compounds with strong and long-wave absorption generally have relatively large pi systems which lead to stronger interactions in the solid state and hence to a higher vaporization temperature. For this reason, it is particularly difficult to invent sublimeable materials for the subcell with long-wave absorption in a tandem cell. To date, apart from particular phthalocyanines, there is no further material class which has a suitable profile of performance properties. It was all the more surprising that the terrylene compounds now discovered are suitable for production of functioning solar cells by vapor deposition processes. In the course of vaporization, there is no decomposition to a degree which prevents use in solar cells.

### SUMMARY OF THE INVENTION

The invention firstly provides a terrylene compound of the general formula (I)

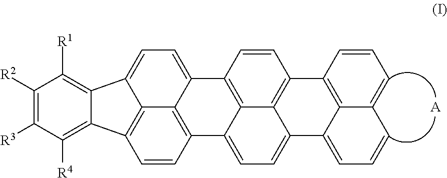

in which
R¹ and R⁴ are each independently selected from hydrogen and in each case unsubstituted or substituted alkyl, aryl, thiophenyl and oligothiophenyl;
R² and R³ are each independently selected from hydrogen and in each case unsubstituted or substituted alkyl, aryl, thiophenyl and oligothiophenyl;
where in each case at least two adjacent radicals selected from the R¹, R², R³ and R⁴ radicals, together with the carbon atoms of the benzene ring to which they are bonded, may also be a fused ring system having 1, 2, 3, 4, 5, 6, 7 or 8 further rings, and
A with the carbon atoms to which it is bonded is a fused monocyclic, dicyclic, tricyclic, tetracyclic, pentacyclic or hexacyclic ring system which optionally bears one or more independently selected substituents.

The invention further provides an organic solar cell with a photoactive region which comprises at least one organic donor material in contact with at least one organic acceptor material, said donor material and said acceptor material forming a donor-acceptor heterojunction, and said photoactive region comprising at least one terrylene compound of the formula (I) as defined above and hereinafter.

The invention further provides processes for preparing the terrylene compounds of the formula (I).

The invention further provides for the use of at least one terrylene compound of the formula (I) as defined above and hereinafter as an electron donor material in organic solar cells.

### DESCRIPTION OF FIGURES

FIG. 1 shows the IPCE of the terrylene compound from example 1.

FIG. 2 shows the basic structure of an inventive tandem cell.

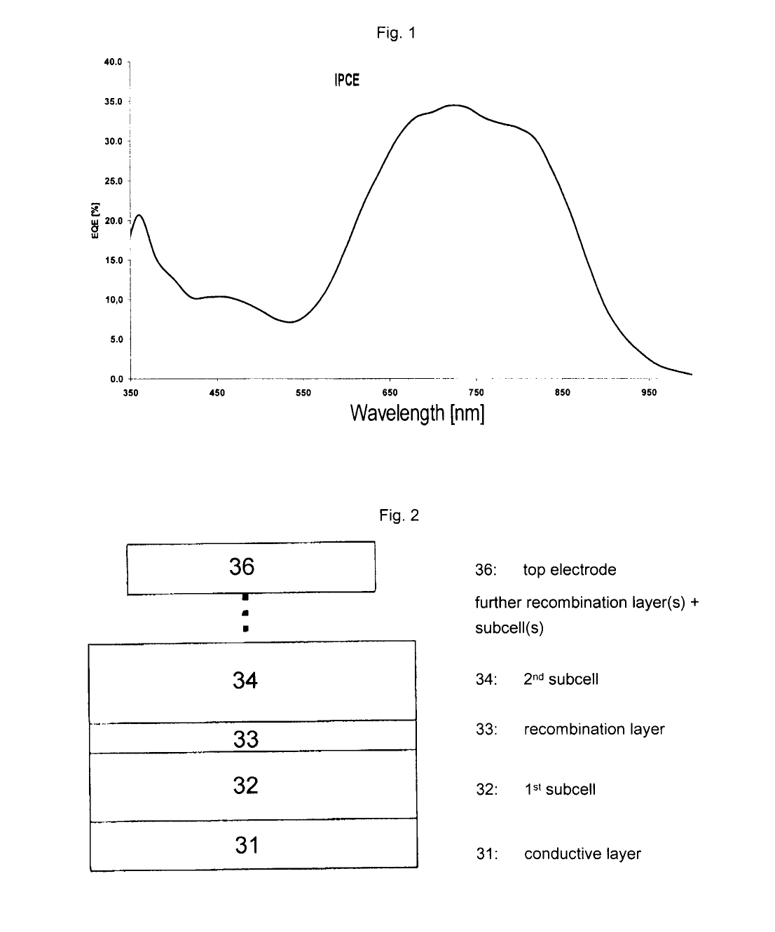

### DESCRIPTION OF THE INVENTION

In the context of the invention, the expression “in each case unsubstituted or substituted alkyl, aryl, thiophenyl or oligothiophenyl” represents unsubstituted or substituted alkyl, unsubstituted or substituted aryl, unsubstituted or substituted thiophenyl or unsubstituted or substituted oligothiophenyl.

In the context of the present invention, the expression “alkyl” comprises straight-chain or branched alkyl. Alkyl is preferably C₁-C₃₀-alkyl, especially C₁-C₂₀-alkyl and most preferably C₁-C₁₂-alkyl. Examples of alkyl groups are especially methyl, ethyl, n-propyl, isopropyl, n-butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

Substituted alkyl groups may, depending on the length of the alkyl chain, have one or more (e.g. 1, 2, 3, 4, 5 or more than 5) substituents. These are preferably selected independently from cycloalkyl and aryl.

Aryl-substituted alkyl radicals (“aralkyl” or “arylalkyl”) have at least one unsubstituted or substituted aryl group as defined hereinafter. Each aryl group has preferably 6 to 14, more preferably 6 to 10, carbon atoms and the alkyl moiety in aralkyl has preferably 1 to 30, more preferably 1 to 12, carbon atoms. Arylalkyl is, for example, phenyl-C₁-C₁₀-alkyl, preferably phenyl-C₁-C₄-alkyl, for example benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl, 4-phenbut-2-yl, 1-(phenmeth)eth-1-yl, 1-(phenmethyl)-1-(methyl)eth-1-yl or 1-(phenmethyl)-1-(methyl)prop-1-yl; preferably benzyl and 2-phenethyl.

In the context of the invention, “cycloalkyl” denotes a cycloaliphatic radical having preferably 3 to 10, more preferably 5 to 8, carbon atoms. Examples of cycloalkyl groups are especially cyclopropyl, cyclobutyl, cyclopentyl, cyclohexyl, cycloheptyl or cyclooctyl.

Substituted cycloalkyl groups may, depending on the ring size, have one or more (e.g. 1, 2, 3, 4, 5 or more than 5) substituents. These are preferably each independently selected from alkyl, cycloalkyl and aryl. In the case of substitution, the cycloalkyl groups preferably bear one or more, for example one, two, three, four or five, C₁-C₆-alkyl groups. Examples of substituted cycloalkyl groups are especially 2- and 3-methylcyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-sec-butylcyclohexyl, 2-, 3- and 4-tert-butylcyclohexyl, 2-, 3- and 4-methylcycloheptyl, 2-, 3- and 4-ethylcycloheptyl, 2-, 3- and 4-propylcycloheptyl, 2-, 3- and 4-isopropylcycloheptyl, 2-, 3- and 4-butylcycloheptyl, 2-, 3- and 4-sec-butylcycloheptyl, 2-, 3- and 4-tert-butylcycloheptyl, 2-, 3-, 4- and 5-methyl-cyclooctyl, 2-, 3-, 4- and 5-ethylcyclooctyl, 2-, 3-, 4- and 5-propylcyclooctyl.

In the context of the present invention, the expression “aryl” comprises mono- or polycyclic aromatic hydrocarbon radicals, having typically 6 to 18, preferably 6 to 14, more preferably 6 to 10, carbon atoms. Examples of aryl are especially phenyl, naphthyl, indenyl, fluorenyl, anthracenyl, phenanthrenyl, naphthacenyl, chrysenyl, pyrenyl, etc., and especially phenyl or naphthyl.

Substituted aryls may, depending on the number and size of their ring systems, have one or more (e.g. 1, 2, 3, 4, 5 or more than 5) substituents. These are preferably each independently selected from alkyl, cycloalkyl and aryl. The alkyl, cycloalkyl and aryl substituents on the aryl may in turn be unsubstituted or substituted. Reference is made to the substituents mentioned above for these groups. One example of substituted aryl is substituted phenyl which generally bears 1, 2, 3, 4 or 5, preferably 1, 2 or 3, substituents.

Substituted aryl is preferably aryl substituted by at least one alkyl group (“alkaryl”). Each alkyl group has typically 1 to 30, preferably 1 to 12, carbon atoms, and the aryl moiety in alkaryl has 6 to 14, preferably 6 to 10, carbon atoms. Alkaryl groups may, depending on the size of the aromatic ring system, have one or more (e.g. 1, 2, 3, 4, 5, 6, 7, 8, 9 or more than 9) alkyl substituents. The alkyl substituents may be unsubstituted or substituted. In this regard, reference is made to the above statements regarding unsubstituted and substituted alkyl. In a preferred embodiment, the alkaryl groups have exclusively unsubstituted alkyl substituents. Alkaryl is preferably phenyl which bears 1, 2, 3, 4 or 5, preferably 1, 2 or 3, more preferably 1 or 2, alkyl substituents having 1 to 12 carbon atoms.

Examples of alkaryl radicals are 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl.

In the context of the present invention, the expression “heteroaryl” (hetaryl) comprises heteroaromatic, mono- or polycyclic groups. In addition to the ring carbon atoms, these have 1, 2, 3, 4 or more than 4 ring heteroatoms. The heteroatoms are preferably selected from oxygen, nitrogen and sulfur. The hetaryl groups have preferably 5 to 18, e.g. 5, 6, 8, 9, 10, 11, 12, 13 or 14, ring atoms.

“Thiophenyl”, also referred to as thienyl, represents thiophen-2-yl and thiophen-3-yl. Substituted thiophenyl represents thiophenyl which bears 1, 2 or 3 substituents selected from C₁-C₈-alkyl, preferably C₁-C₄-alkyl.

The oligothiophenyl group bears typically 1 to 5 thiophenediyl groups bonded to one another via a single bond and, as a terminal group, a thiophenyl group. The thiophenyl group may be unsubstituted or substituted. Each thiophenediyl group may be unsubstituted or substituted. Suitable substituents are selected from C₁-C₈-alkyl.

Examples of oligothiophenyl groups are

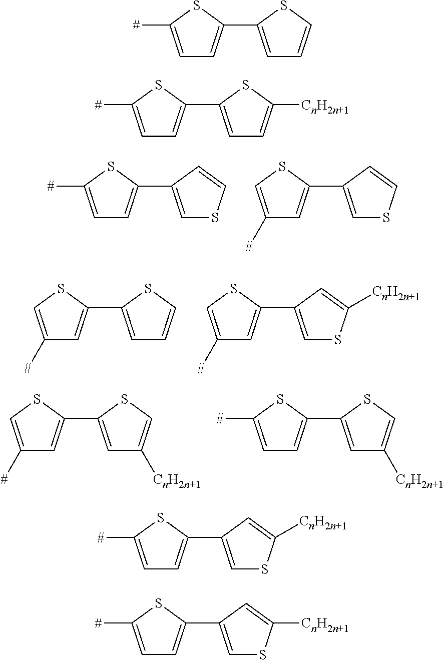

in which # represents a bonding site to the rest of the molecule and n is 1, 2, 3, 4, 5, 6, 7 or 8.

### R¹ and R⁴ Radicals

In the compounds of the general formula (I), the R¹ and R⁴ radicals may both have the same definition or may have different definitions. R¹ and R⁴ preferably have the same definition.

Preferably, R¹ and R⁴ are each independently selected from hydrogen, unsubstituted alkyl, aralkyl, unsubstituted aryl, alkaryl, thiophenyl and oligothiophenyl, where the two latter radicals may bear one or more alkyl substituents.

In a specific embodiment, R¹ and/or R⁴ are each unsubstituted or substituted C1- to C₃₀-alkyl. Preferably, R¹ and/or R⁴ are each unsubstituted or substituted C₁- to C₁₂-alkyl. More preferably, R¹ and/or R⁴ are each unsubstituted linear C1- to C₁₂-alkyl, especially unsubstituted linear C4- to C₁₂-alkyl, such as n-octyl, n-nonyl, n-decyl, n-undecyl, n-dodecyl. R¹ and R⁴ are especially unsubstituted or substituted C₁- to C₁₂-alkyl, more especially unsubstituted linear C₁- to C₁₂-alkyl, such as methyl, ethyl, n-propyl, n-butyl, n-octyl, n-nonyl, n-decyl, n-undecyl, n-dodecyl.

In a further specific embodiment, R¹ and/or R⁴ are each thiophenyl which may bear 1, 2, or 3 C₁-C₈-alkyl substituents. In a further specific embodiment, R¹ and/or R⁴ are each oligothiophenyl which may bear 1, 2, 3, 4 or more than 4 C₁-C₈-alkyl substituents.

In a further embodiment, R¹ and/or R⁴ are each alkaryl.

Preferably, R¹ and R⁴ are each independently selected from hydrogen and groups of the general formulae (III.1) to (III.12)

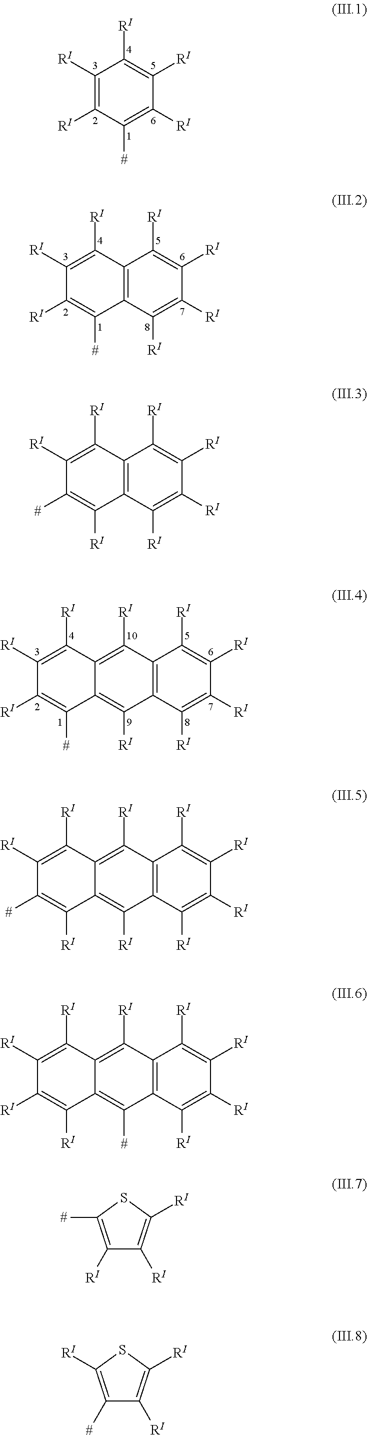

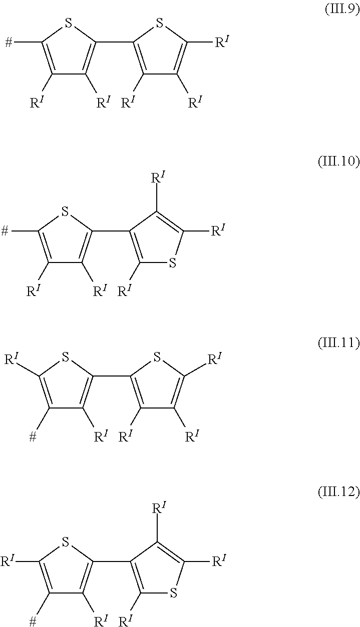

in which
# represents the bonding site to the benzene ring, and
R^{I} is in each case independently selected from hydrogen, unsubstituted alkyl, alkaryl, arylalkyl and alkaryl.

(The numbering of the aromatic ring systems is used hereinafter to specify the position of the substituents.)

Preferably, 0, 1 or 2 of the R^{I} radicals in the groups of the formula (III.1) have a definition other than hydrogen. Monosubstituted groups of the formula (III.1) preferably have an R^{I} radical in the 4 position. Disubstituted groups of the formula (III.1) preferably have two R^{I} radicals in the 3 position and in the 5 position.

Preferably, 0, 1, 2, 3 or 4 of the R^{I} radicals in the groups of the formula (III.2) have a definition other than hydrogen. More preferably, 0 or 1 of the R^{I} radicals in the groups of the formula (III.2) have a definition other than hydrogen. Monosubstituted groups of the formula (III.2) preferably have an R^{I} radical in the 4 position.

Preferably, 0, 1, 2, 3 or 4 of the R^{I} radicals in the groups of the formula (III.3) have a definition other than hydrogen. More preferably, 0 or 1 of the R^{I} radicals in the groups of the formula (III.3) have a definition other than hydrogen.

Preferably, 0, 1, 2, 3, 4, 5 or 6 of the R^{I} radicals in the groups of the formula (III.4) have a definition other than hydrogen. More preferably, 0 or 1 of the R^{I} radicals in the groups of the formula (III.4) have a definition other than hydrogen.

Preferably, 0, 1, 2, 3, 4, 5 or 6 of the R^{I} radicals in the groups of the formula (III.5) have a definition other than hydrogen. More preferably, 0 or 1 of the R^{I} radicals in the groups of the formula (III.5) have a definition other than hydrogen.

Preferably, 0, 1, 2, 3, 4, 5 or 6 of the R^{I} radicals in the groups of the formula (III.6) have a definition other than hydrogen. More preferably, 0 or 1 of the R^{I} radicals in the groups of the formula (III.6) have a definition other than hydrogen.

Preferably, 0, 1 or 2 of the R^{I} radicals in the groups of the formula (III.7) have a definition other than hydrogen. More preferably, 0 or 1 of the R^{I} radicals in the groups of the formula (III.7) have a definition other than hydrogen.

Preferably, 0, 1 or 2 of the R^{I} radicals in the groups of the formula (III.8) have a definition other than hydrogen. More preferably, 0 or 1 of the R^{I} radicals in the groups of the formula (III.8) have a definition other than hydrogen.

Preferably, 0, 1, 2, 3 or 4 of the R^{I} radicals in the groups of the formulae (III.9) to (III.12) have a definition other than hydrogen. Preferably, 0, 1 or 2 of the R^{I} radicals on each thiophene ring have a definition other than hydrogen. More preferably, 0 or 1 of the R^{I} radicals in the groups of the formulae (III.9) to (III.12) have a definition other than hydrogen.

Preferably, the R^{I} radicals in the (III.1) to (III.12) groups are each independently selected from hydrogen and C₁-C₂₀-alkyl. More preferably, the R^{I} radicals in the (III.1) to (III.12) groups are each independently selected from hydrogen and C1- to C₁₂-alkyl.

In a specific embodiment, the R^{I} radicals which have a definition other than hydrogen in the (III.1) to (III.12) groups are selected from unsubstituted C₁- to C₄-alkyl groups such as methyl, ethyl, n-propyl, isopropyl, n-butyl and tert-butyl.

In a further specific embodiment, the R^{I} radicals which have a definition other than hydrogen in the (III.1) to (III.12) groups are selected from unsubstituted linear C1- to C₁₂-alkyl groups such as methyl, ethyl, n-propyl, n-octyl, n-nonyl, n-decyl, n-undecyl, n-dodecyl.

More particularly, R¹ and R⁴ are each independently selected from hydrogen and groups of the general formulae (III.1a), (III.1b), (III.1c), (III.1d), (III.2a), (III.4a), (III.7a), (III.7b), (III.8a), (III.8b), (III.9a), (III.9b), (III.10a), (III.10b), (III.11a), (III.11b), (III.12a) or (III.12b):

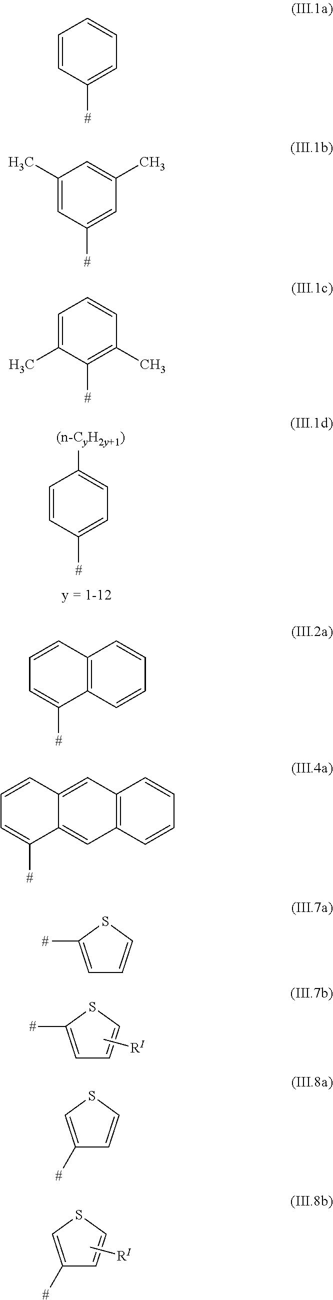

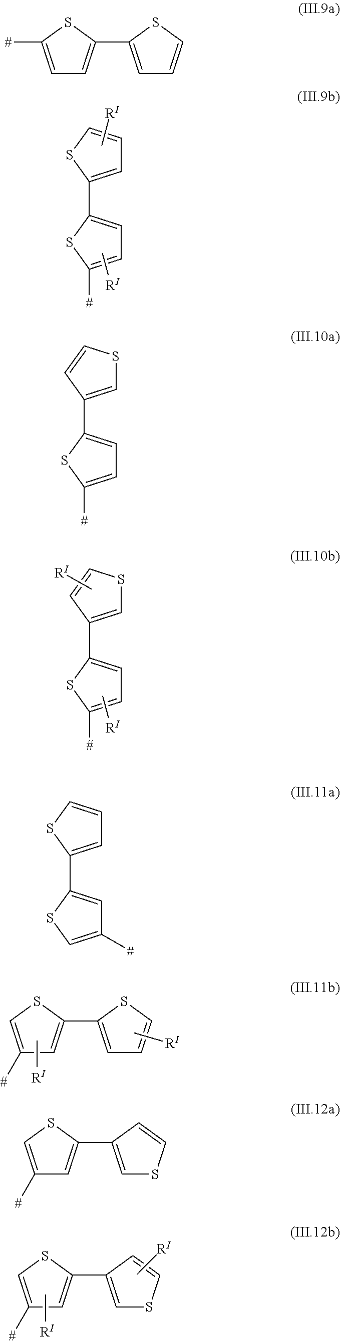

in which
# represents the bonding site to the benzene ring; and
R^{I} is hydrogen or C₁-C₈-alkyl.

More particularly, the R¹ and R⁴ groups are each independently selected from hydrogen, phenyl, thiophen-2-yl, thiophen-3-yl, 5-methylthiophen-2-yl. Most preferably, the R¹ and R⁴ groups are each independently selected from phenyl, thiophen-2-yl and 5-methylthiophen-2-yl. R¹ and R⁴ are especially both thiophen-2-yl or both phenyl. Both are especially hydrogen. Both are especially 5-methylthiophen-2-yl.

### R² and R³ Radicals

In the compounds of the general formula (I), the R² and R³ radicals may both have the same definition or may have different definitions. R² and R³ preferably have the same definition.

Preferably, R² and R³ are each independently selected from hydrogen, unsubstituted alkyl, aralkyl, unsubstituted aryl, alkaryl, thiophenyl and oligothiophenyl, where the two latter radicals may bear one or more alkyl substituents.

In a specific embodiment, R² and/or R³ are each unsubstituted or substituted C₁- to C₃₀-alkyl. Preferably, R² and/or R³ are each unsubstituted or substituted C₁- to C₁₂-alkyl. More preferably, R² and/or R³ are each unsubstituted linear C₁- to C₁₂-alkyl, especially unsubstituted linear C₄- to C₁₂-alkyl, such as n-octyl, n-nonyl, n-decyl, n-undecyl, n-dodecyl. R² and R³ are especially unsubstituted or substituted C₁- to C₁₂-alkyl, more especially unsubstituted linear C₁- to C₁₂-alkyl, such as methyl, ethyl, n-propyl, n-octyl, n-nonyl, n-decyl, n-undecyl, n-dodecyl.

In a further embodiment, R² and/or R³ are each alkaryl.

In a further embodiment, R² and/or R³ are each thiophenyl which may bear 1, 2, or 3 C₁-C₈-alkyl substituents. In a further specific embodiment, R² and/or R³ are each oligothiophenyl which may bear 1, 2, 3, 4 or more than 4 C₁-C₈-alkyl substituents.

In a specific embodiment, the R² and R³ groups in the compounds of the general formula (I) are each independently selected from hydrogen and groups of the general formulae (IV.1) to (IV.12)

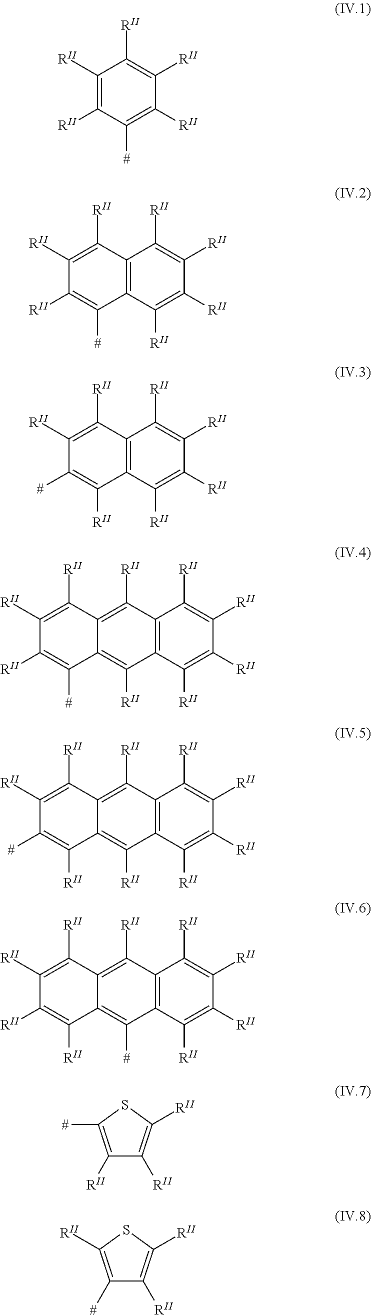

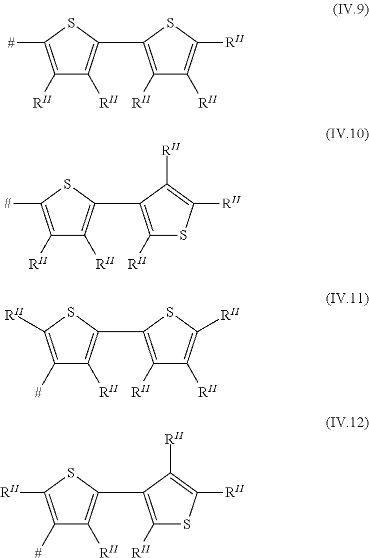

in which
# represents the bonding site to the benzene ring, and
R^{II} is in each case independently selected from hydrogen, unsubstituted alkyl, aryl, aralkyl and alkaryl.

Preferably, 0, 1 or 2 of the R^{II} radicals in the groups of the formula (IV.1) have a definition other than hydrogen. Monosubstituted groups of the formula (IV.1) preferably have an R^{II} radical in the 4 position. Disubstituted groups of the formula (IV.1) preferably have two R^{II} radicals in the 3 position and in the 5 position.

Preferably, 0, 1, 2, 3 or 4 of the R^{II} radicals in the groups of the formula (IV.2) have a definition other than hydrogen. More preferably, 0 or 1 of the R^{II} radicals in the groups of the formula (IV.2) have a definition other than hydrogen. Monosubstituted groups of the formula (IV.2) preferably have an R^{II} radical in the 4 position.

Preferably, 0, 1, 2, 3 or 4 of the R^{II} radicals in the groups of the formula (IV.3) have a definition other than hydrogen. More preferably, 0 or 1 of the R^{II} radicals in the groups of the formula (IV.3) have a definition other than hydrogen.

Preferably, 0, 1, 2, 3, 4, 5 or 6 of the R^{II} radicals in the groups of the formula (IV.4) have a definition other than hydrogen. More preferably, 0 or 1 of the R^{II} radicals in the groups of the formula (IV.4) have a definition other than hydrogen.

Preferably, 0, 1, 2, 3, 4, 5 or 6 of the R^{II} radicals in the groups of the formula (IV.5) have a definition other than hydrogen. More preferably, 0 or 1 of the R^{II} radicals in the groups of the formula (IV.5) have a definition other than hydrogen.

Preferably, 0, 1, 2, 3, 4, 5 or 6 of the R^{II} radicals in the groups of the formula (IV.6) have a definition other than hydrogen. More preferably, 0 or 1 of the R^{II} radicals in the groups of the formula (IV.6) have a definition other than hydrogen.

Preferably, 0, 1 or 2 of the R^{II} radicals in the groups of the formula (IV.7) have a definition other than hydrogen. More preferably, 0 or 1 of the R^{II} radicals in the groups of the formula (IV.7) have a definition other than hydrogen.

Preferably, 0, 1 or 2 of the R^{II} radicals in the groups of the formula (IV.8) have a definition other than hydrogen. More preferably, 0 or 1 of the R^{II} radicals in the groups of the formula (IV.8) have a definition other than hydrogen.

Preferably, 0, 1, 2, 3 or 4 of the R^{II} radicals in the groups of the formulae (IV.9) to (IV.12) have a definition other than hydrogen. Preferably, 0, 1 or 2 of the RU radicals on each thiophene ring have a definition other than hydrogen. More preferably, 0 or 1 of the R^{II} radicals in the groups of the formulae (IV.9) to (IV.12) have a definition other than hydrogen.

In a specific embodiment, the R^{II} radicals which have a definition other than hydrogen in the (IV.1) to (IV.12) groups are selected from unsubstituted C₁- to C₄-alkyl groups such as methyl, ethyl, n-propyl, isopropyl, n-butyl and tert-butyl.

In a further specific embodiment, the R^{II} radicals which have a definition other than hydrogen in the (IV.1) to (IV.12) groups are selected from unsubstituted linear C1- to C₁₂-alkyl groups such as methyl, ethyl, n-propyl, n-octyl, n-nonyl, n-decyl, n-undecyl, n-dodecyl.

More particularly, R² and R³ are each independently selected from hydrogen and groups of the general formulae (IV.1a), (IV.1b), (IV.1c), (IV.1d), (IV.2a), (IV.4a), (IV.7a), (IV.7b), (IV.8a), (IV.8b), (IV.9a), (IV.9b), (IV.10a), (IV.10b), (IV.11a), (IV.11b), (IV.12a) or (IV.12b):

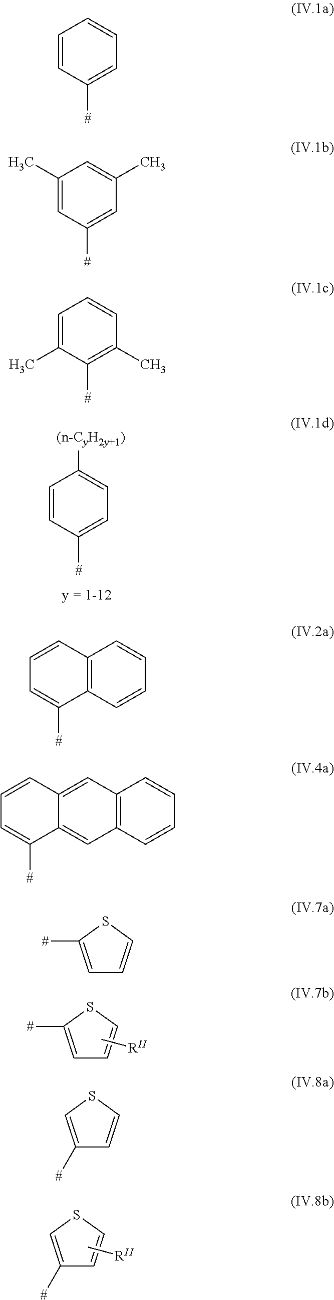

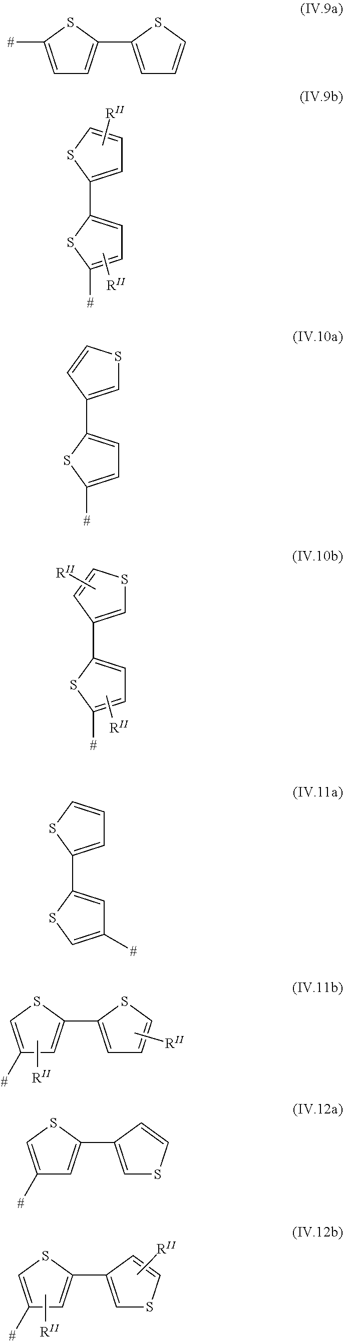

in which
# represents the bonding site to the benzene ring; and
R^{II} is hydrogen or C₁-C₈-alkyl.

More particularly, the R² and R³ groups are each independently selected from hydrogen and phenyl. R² and R³ are especially both hydrogen or both phenyl.

### Fused R² and R³ Radicals

In a specific embodiment, the two R² and R³ radicals in the compounds of the general formula (I) together with the carbon atoms of the benzene ring to which they are bonded are a fused ring system having 1, 2, 3 or 4 further rings.

Preferably, R² and R³ in the compounds of the general formula (I) together are a group selected from groups of the general formulae (V.1), (V.2) and (V.3)

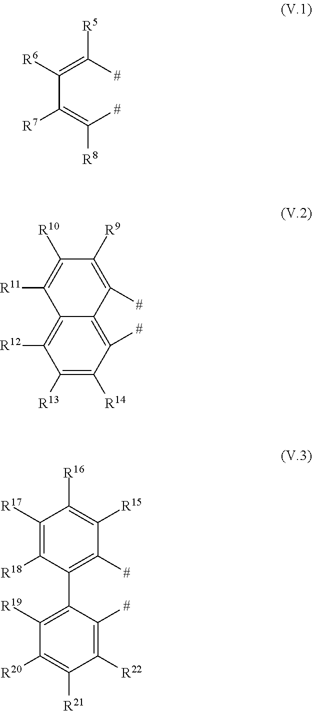

in which
# in each case represents a bonding site to the benzene ring;
R⁵, R⁶, R⁷ and R⁸ are each independently hydrogen, C₁-C₂₀-alkyl or phenyl, where phenyl may be unsubstituted or may bear 1, 2 or 3 R^{a} radicals;
R⁹, R¹⁰, R¹¹, R¹², R¹³ and R¹⁴ are each independently hydrogen, C₁-C₂₀-alkyl or phenyl, where phenyl may be unsubstituted or may bear 1, 2 or 3 R^{a} radicals; and
R¹⁵, R¹⁶, R¹⁷, R¹⁸, R¹⁹, R²⁰, R²¹ and R²² are each independently hydrogen, C₁-C₂₀-alkyl or phenyl, where phenyl may be unsubstituted or may bear 1, 2 or 3 R^{a} radicals, in which
R^{a} is C₁-C₁₀-alkyl.

In a preferred embodiment, R² and R³ together are a group of the formula (V.1). Preferably, R⁵, R⁶, R⁷ and R⁸ in the groups (V.1) are each hydrogen.

Preferably, 0, 1, 2, 3 or 4 of the R³ radicals in the groups of the formulae (V.2) and (V.3) have a definition other than hydrogen.

Preferably, the R⁵ to R²² radicals in the (V.2) and (V.3) groups are each independently selected from hydrogen, C₁- to C₂₀-alkyl and phenyl.

More preferably, the R⁵ to R²² radicals in the (V.1) to (V.3) groups are each independently selected from hydrogen, C₁- to C₁₂-alkyl and phenyl.

In a specific embodiment, the R⁵ to R²² radicals which have a definition other than hydrogen in the (V.1) to (V.3) groups are selected from unsubstituted C₁- to C₄-alkyl groups, such as methyl, ethyl, n-propyl, isopropyl, n-butyl and tert-butyl.

In a further specific embodiment, the R⁵ to R²² radicals which have a definition other than hydrogen in the (V.1) to (V.3) groups are selected from unsubstituted linear C4- to C₁₂-alkyl groups such as n-octyl, n-nonyl, n-decyl, n-undecyl, n-dodecyl.

More preferably, R² and R³ together are a (V.1), (V.2) or (V.3) group, in which the R⁵ to R²² radicals are all hydrogen.

### Fused A Groups

Preferably, the A groups in the compounds of the general formula (I) are selected from groups of the general formulae (VI.1), (VI.2), (VI.3), (VI.4), (VI.5), (VI.6) and (IV.7)

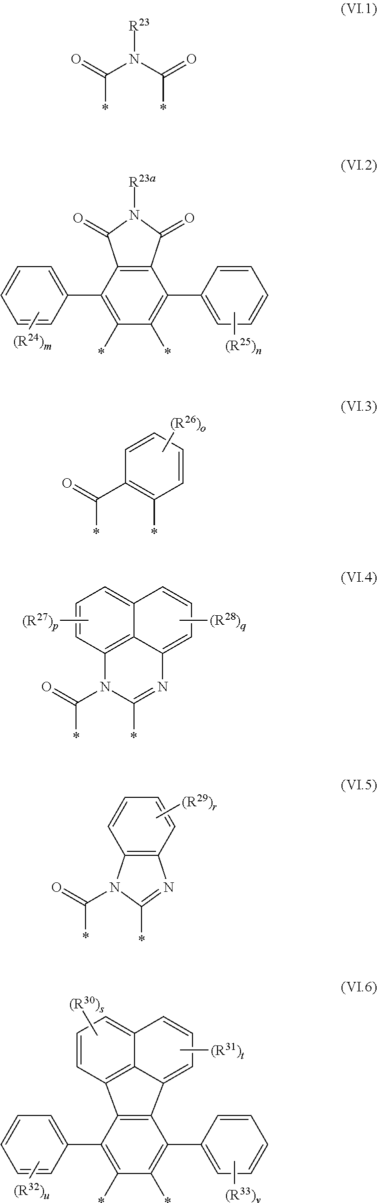

in which
in each case represents the bonding site to the terrylene base skeleton,
R²³ is hydrogen, alkyl, aryl, alkaryl or aralkyl,
R^{23a} is hydrogen, alkyl, aryl, alkaryl or aralkyl,
R²⁴, R²⁵ are each independently alkyl, aryl, alkaryl or aralkyl, where two R²⁴ and/or R²⁵ radicals bonded to adjacent carbon atoms may also be a fused-on benzene ring,
R²⁶ is alkyl, aryl, alkaryl or aralkyl, where two R²⁶ radicals bonded to adjacent carbon atoms may be a fused-on benzene ring,
R²⁷, R²⁸ are each independently alkyl, aryl, alkaryl or aralkyl, where two R²⁷ and/or R²⁸ radicals bonded to adjacent carbon atoms may also be a fused-on benzene ring,
R²⁹ is alkyl, aryl, alkaryl or aralkyl, where two R²⁹ radicals bonded to adjacent carbon atoms may also be a fused-on benzene ring,
R³⁰, R³¹ are each independently cyano, alkyl, aryl, alkaryl or aralkyl,
R³², R³³ are each independently cyano, alkyl, aryl, alkaryl or aralkyl,
m, n, u, v are each independently 0, 1, 2 or 3, and
o, p, q, r, s, t are each independently 0, 1 or 2.
   The A group is more preferably selected from groups of the general formulae (VI.1), (VI.2), (VI.3), (VI.4), (VI.5) and (VI.6), in which
   in each case represents the bonding site to the terrylene base skeleton,
R²³, R^{23a} are each hydrogen, C₁-C₂₀-alkyl, C₆-C₁₄-aryl-C₁-C₂₀-alkyl, C₆-C₁₄-aryl or C₁-C₂₀-alkyl-C₆-C₁₄-aryl,
R²⁴, R²⁵ are each independently C₁-C₂₀-alkyl or phenyl, where phenyl may be unsubstituted or may bear 1, 2 or 3 R^{b} radicals, and where two R²⁴ and/or R²⁵ radicals bonded to adjacent carbon atoms may also be a fused-on benzene ring,
R²⁶ is C₁-C₂₀-alkyl or phenyl, where phenyl may be unsubstituted or may bear 1, 2 or 3 R^{b} radicals, and where two R²⁶ radicals bonded to adjacent carbon atoms may be a fused-on benzene ring,
R²⁷, R²⁸ are each independently C₁-C₂₀-alkyl or phenyl, where phenyl may be unsubstituted or may bear 1, 2 or 3 R^{b} radicals, and where two R²⁷ and/or R²⁸ radicals bonded to adjacent carbon atoms may also be a fused-on benzene ring,
R²⁹ is C₁-C₂₀-alkyl or phenyl, where phenyl may be unsubstituted or may bear 1, 2 or 3 R^{b} radicals, and where two R²⁹ radicals bonded to adjacent carbon atoms may also be a fused-on benzene ring,
R³⁰, R³¹ are each independently C₁-C₂₀-alkyl, cyano or phenyl, where phenyl may be unsubstituted or may bear 1, 2 or 3 R^{b} radicals,
R³², R³³ are each independently C₁-C₂₀-alkyl, cyano or phenyl, where phenyl may be unsubstituted or may bear 1, 2 or 3 R^{b} radicals,
m, n, u, v are each independently 0, 1, 2 or 3,
o, p, q, r, s, t are each 0, 1 or 2,
R^{b} is C₁-C₂₀-alkyl, where different R^{b} radicals may each have identical or different definitions.

More preferably, A is a group of the formula (VI.1) in which R²³ is hydrogen, C₁-C₂₀-alkyl or phenyl, where phenyl is unsubstituted or bears 1, 2 or 3 C₁-C₂₀-alkyl radicals. More particularly, A is a group of the formula (VI.1) in which R²³ is phenyl. More particularly, A is additionally a group of the formula (VI.1) in which R²³ is methyl.

More preferably, A is a group of the formula (VI.6a)

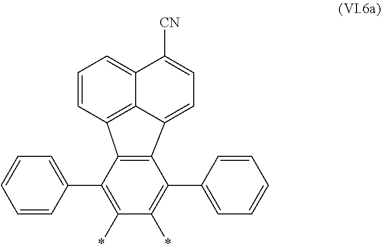

A specific embodiment is a terrylene compound of the formula (I) in which
R¹ and R⁴ are both phenyl,
R² and R³ together are a radical of the formula (V.1) in which R⁵, R⁶, R⁷ and R⁸ are each hydrogen, and
A is a radical of the formula (VI.1) in which R²³ is phenyl or methyl or hydrogen.

Examples of terrylene compounds which are preferentially suitable for use in organic solar cells are reproduced below:

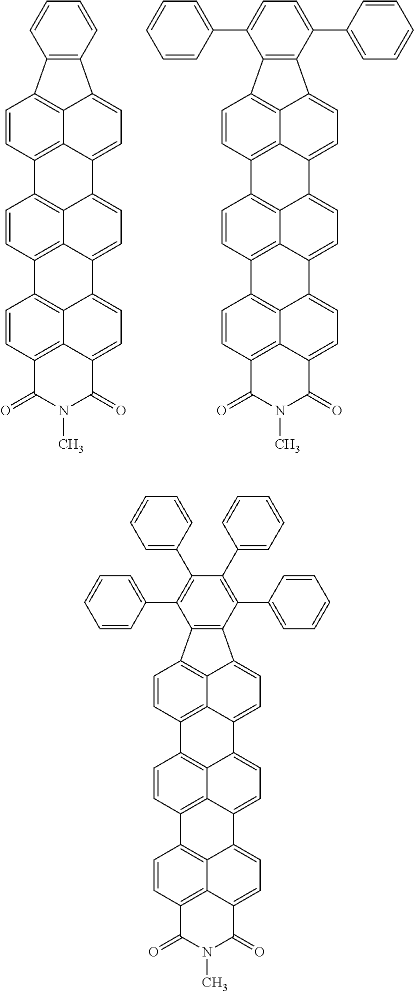

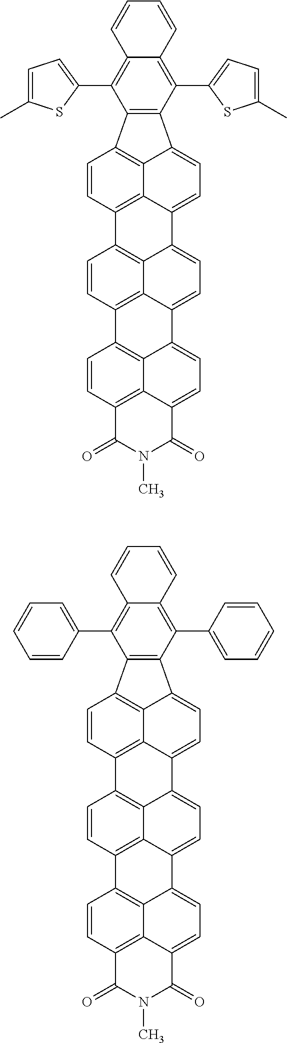

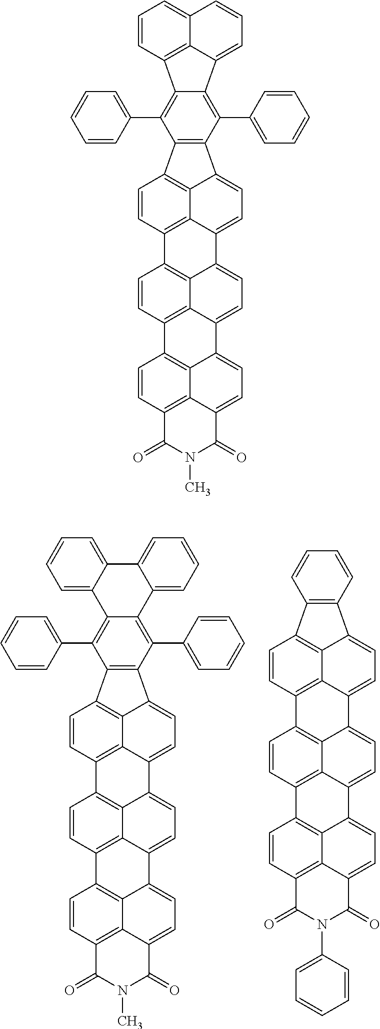

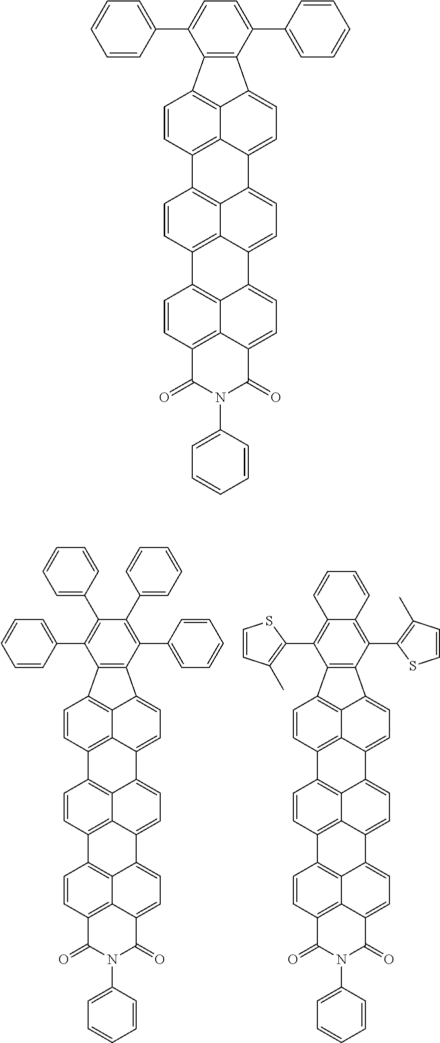

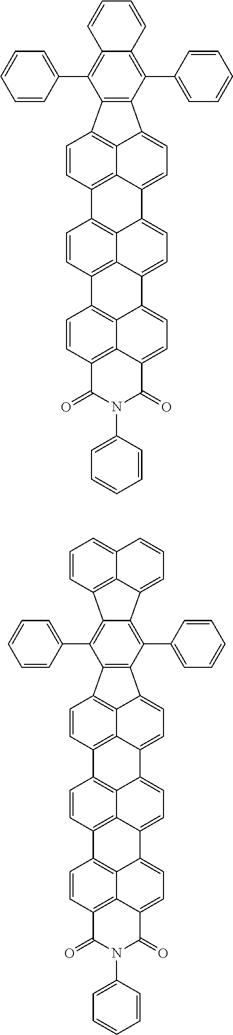

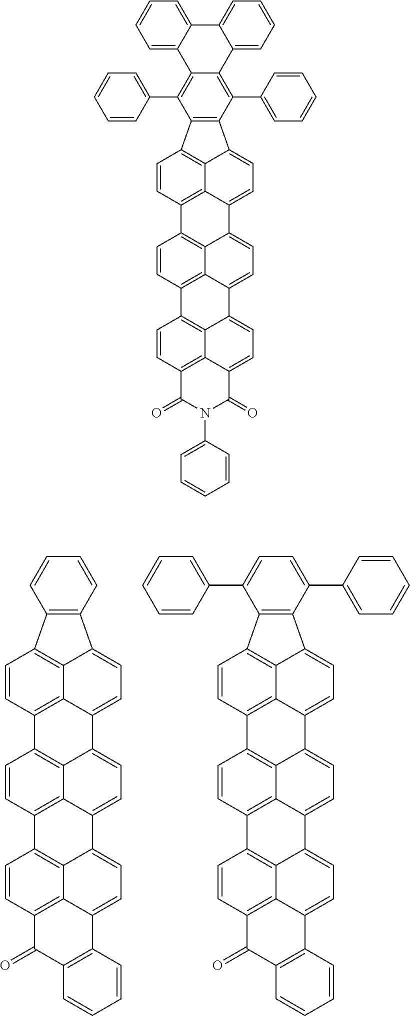

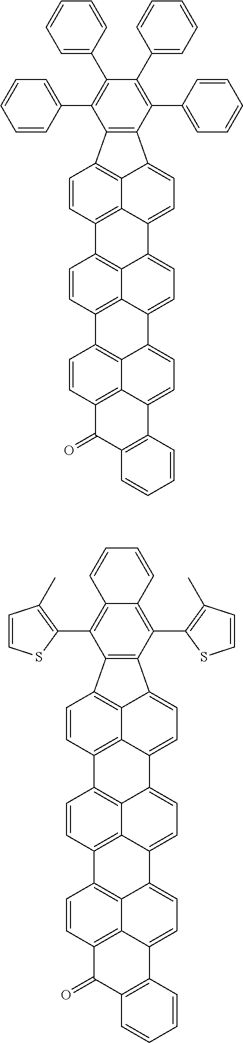

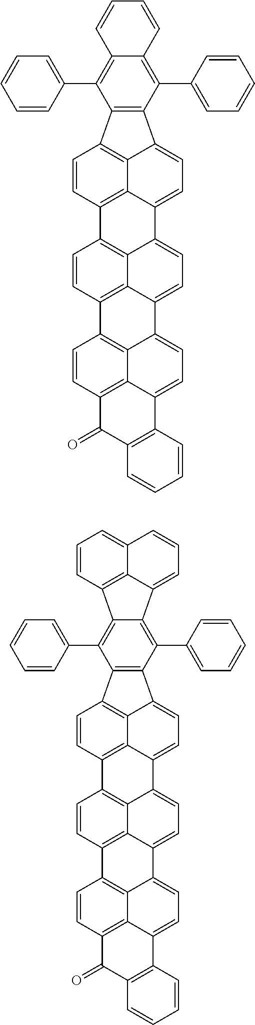

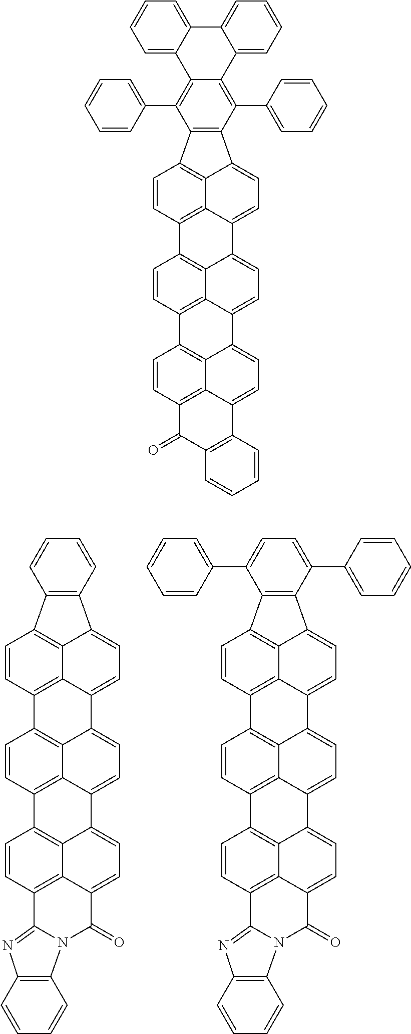

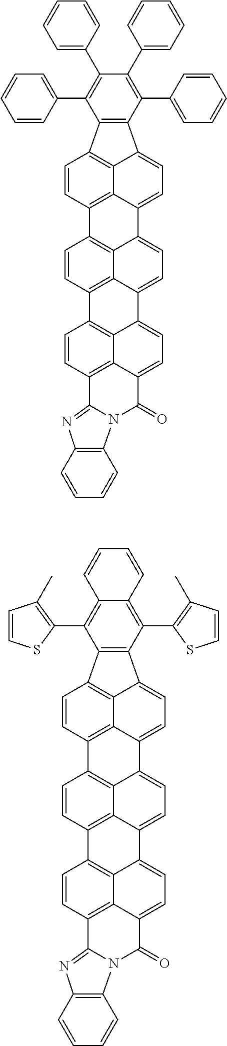

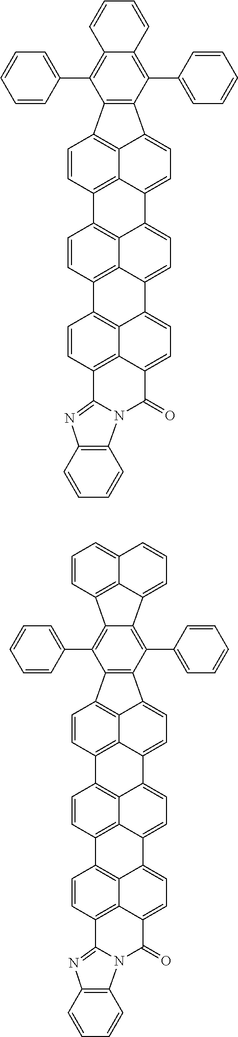

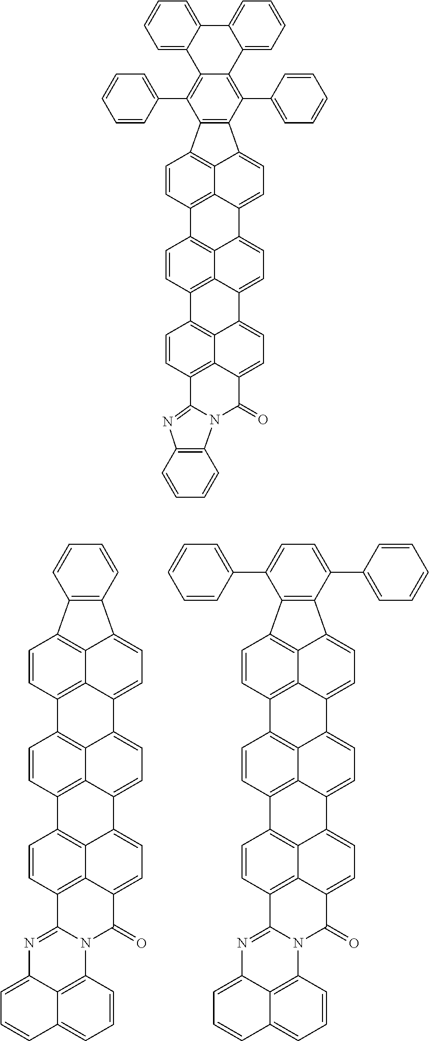

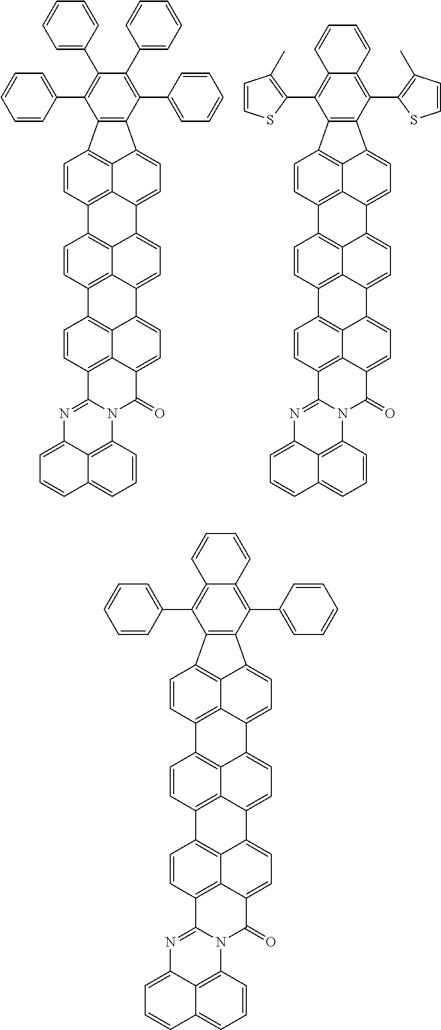

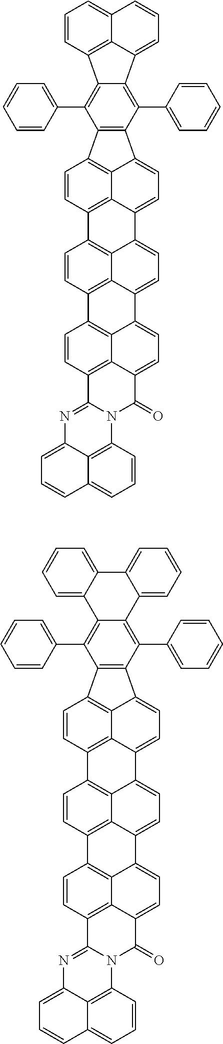

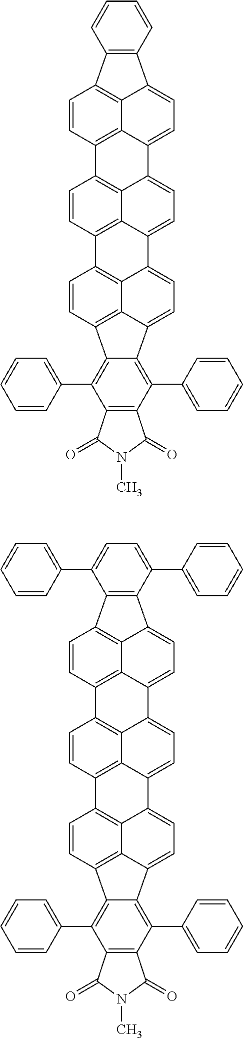

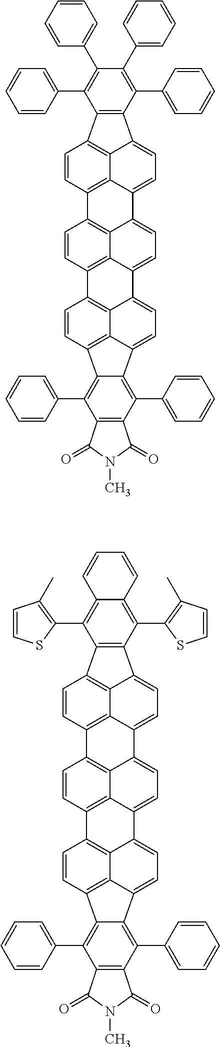

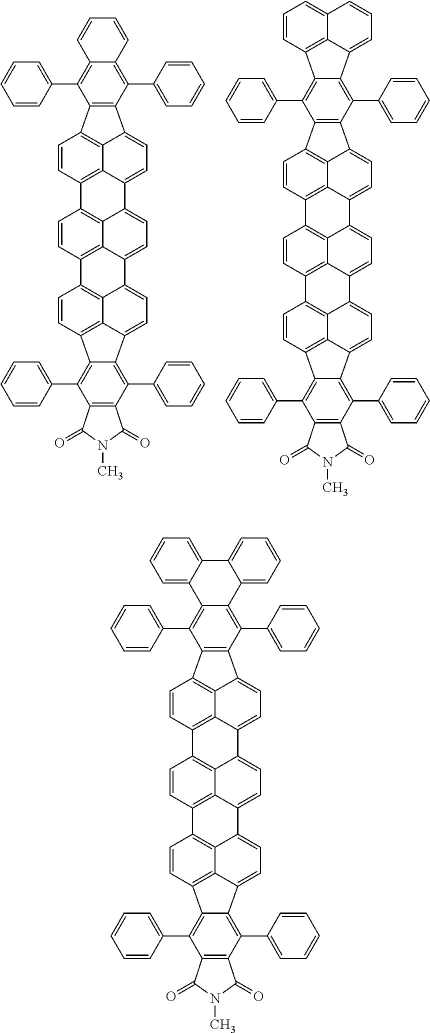

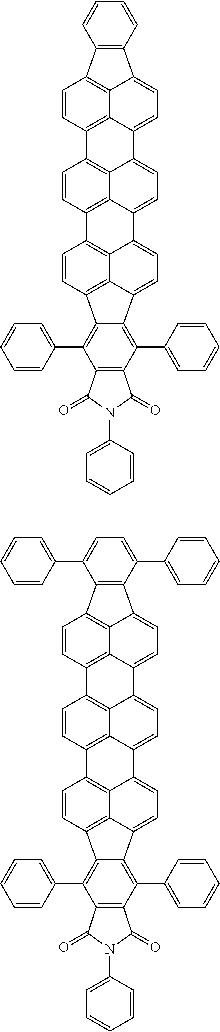

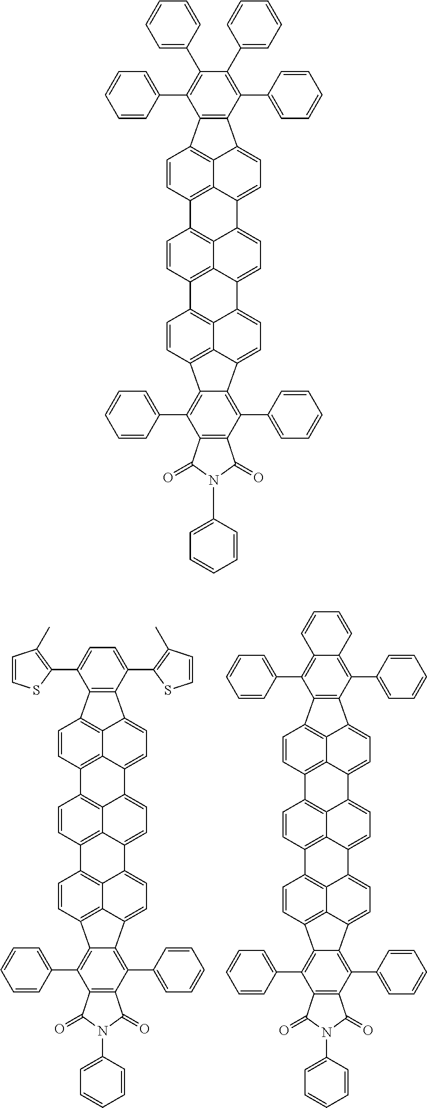

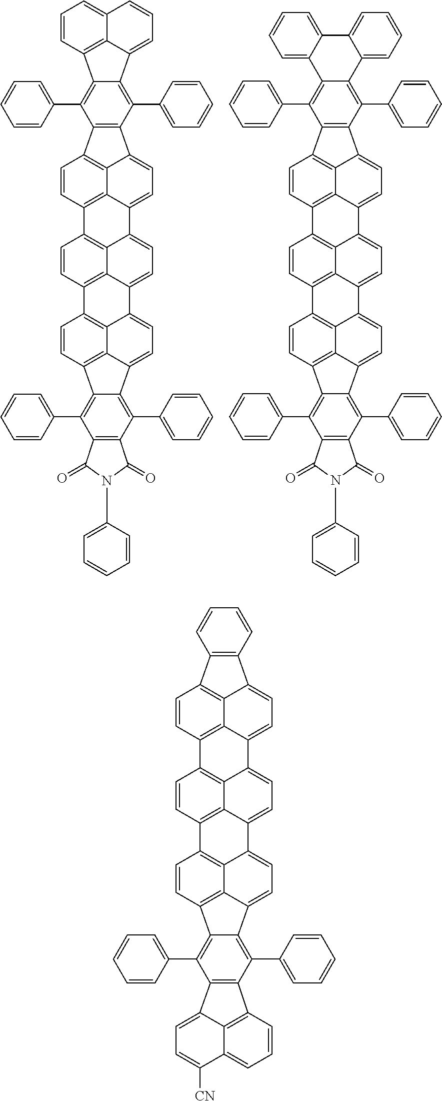

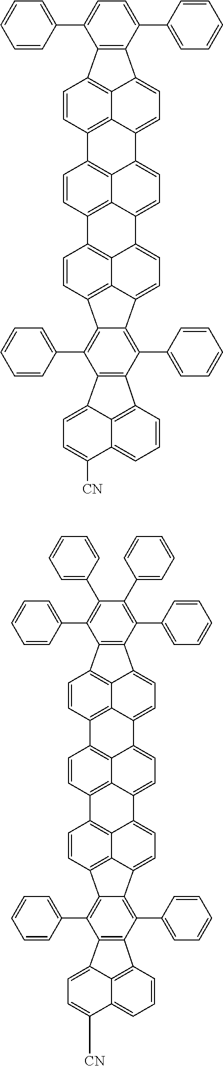

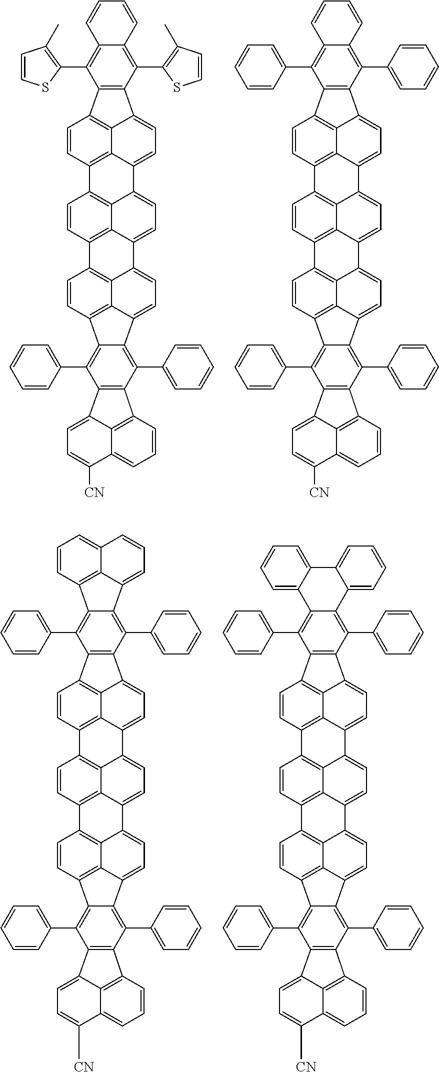

### Synthesis

The inventive compounds I can be synthesized in various ways, for example by [2+1] synthesis from a perylenemonoimide unit and a naphthalene unit (process variant A) or by [1+1+1] synthesis from three naphthalene units (process variant B).

### Process Variant A:

Process variant A comprises two embodiments, which are shown in schemes 1, 2 and 3.

### Process Variant A1:

Process variant A1 is especially suitable for preparation of compounds I in which A is a radical of the formula VI.1.

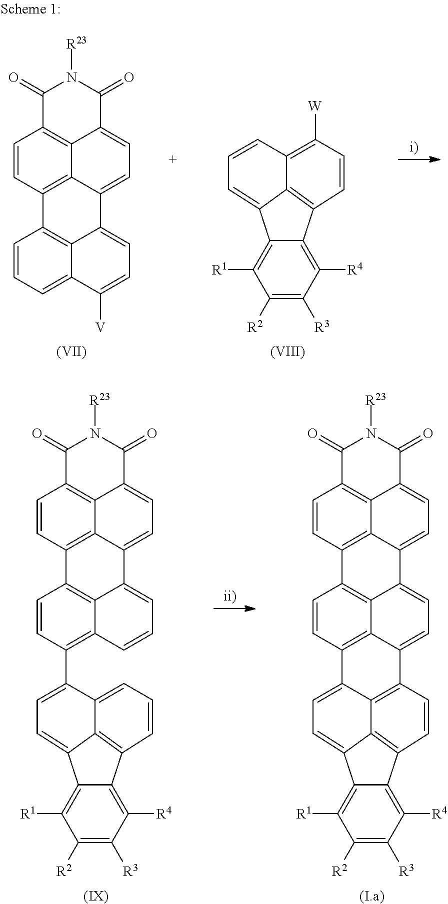

In scheme 1, R¹, R², R³, R⁴ and R²³ each have the definitions specified above, especially the definitions specified as preferred. One of the V and W radicals is chlorine, bromine or iodine, preferably bromine, and the other V or W radical is a B(OR′)(OR″) radical in which R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene which optionally bears 1, 2, 3, 4, 5, 6, 7 or 8 substituents selected from C₁-C₄-alkyl, C₅-C₈-cycloalkyl, C₆-C₁₄-aryl or hetaryl. Preferably, R′ and R″ are each independently hydrogen or C₁-C₄-alkyl or R′ and R″ together are C₂-C₄-alkylene optionally substituted by 1, 2, 3 or 4 C₁-C₄-alkyl groups.

In step i), the molecular skeleton is formed from a halonaphthalene compound VIII with a perylenemonoimideboronic acid derivative VII, preferably under the conditions of a Suzuki coupling. Alternatively, the molecular skeleton is formed from a haloperylene compound VII and a naphthaleneboronic acid derivative VIII, preferably under the conditions of a Suzuki coupling. In other words, the formation is preferably effected in the presence of a platinum metal catalyst and especially in the presence of a palladium catalyst under reaction conditions known per se, as known, for example, from Acc. Chem. Res. 15, p. 178-184 (1982), Chem. Rev. 95, p. 2457-2483 (1995), and the literature cited therein, and from J. Org. Chem. 68, p. 9412 (2003). Suitable catalysts are especially tetrakis(triphenylphosphine)palladium(0), tetrakis(tris-o-tolylphosphine)palladium(0), [1,2-bis(diphenylphosphino)ethane]palladium(II) chloride, [1,1′-bis(diphenylphosphino)ferrocene]palladium(II) chloride, bis(triethylphosphine)palladium(II) chloride, bis(tricyclohexylphosphine)palladium(II) acetate, (2,2′-bipyridyl)palladium(II) chloride, bis(triphenylphosphine)palladium(II) chloride, tris(dibenzylideneacetone)dipalladium(0), 1,5-cyclooctadienepalladium(II) chloride, bis(acetonitrile)palladium(II) chloride and bis(benzonitrile)palladium(II) chloride, preference being given to [1,1-bis(diphenylphosphino)ferrocene]palladium(II) chloride and tetrakis(triphenylphosphine)palladium(0). The amount of catalyst is typically 1 to 20 mol %, especially 1.5 to 5 mol %, based on the boronic acid derivative used in each case.

Preference is given to reacting the boronic acid derivative with the halonaphthalene compound in the presence of an organic solvent, optionally in a mixture with water. Suitable solvents are especially those solvents in which the boronic acid derivative VII and the halonaphthalene compound VIII dissolve completely and the catalysts and bases used at least partially at reaction temperature, such that substantially homogeneous reaction conditions are present.

Examples of suitable solvents are octane, isooctane, nonane, isononane, decane, isodecane, undecane, dodecane, hexadecane and octadecane; cycloheptane, cyclooctane, methylcyclohexane, dimethylcyclohexane, trimethylcyclohexane, ethylcyclohexane, diethylcyclohexane, propylcyclohexane, isopropylcyclohexane, dipropylcyclohexane, butylcyclohexane, tert-butylcyclohexane, methylcycloheptane and methylcyclooctane; toluene, o-, m- and p-xylene, 1,3,5-trimethylbenzene, 1,2,4- and 1,2,3-trimethylbenzene, ethylbenzene, propylbenzene, isopropylbenzene, butylbenzene, isobutylbenzene, tert-butylbenzene and cyclohexylbenzene; naphthalene, decahydronaphthalene, 1- and 2-methylnaphthalene and 1- and 2-ethylnaphthalene, and mixtures of the aforementioned solvents. Very particularly preferred solvents are xylenes such as o-xylene, m-xylene, p-xylene, mesitylene, toluene and mixtures thereof, in particular toluene.

Preference is given to using water as an additional solvent.

Useful bases are preferably the alkali metal salts, especially the sodium and in particular the potassium salts of weak acids, particular preference being given to the carbonates such as sodium carbonate and in particular potassium carbonate. Phosphates such as sodium phosphate or potassium phosphate are also suitable. In general, the amount of base is 1 to 100 mol, especially 5 to 50 mol and in particular 10 to 30 mol, per mole of boronic acid derivative.

The reaction temperature is generally 20 to 180° C., preferably 20 to 120° C.

The boronic acid derivatives can be prepared, for example, by converting the corresponding halogenated aromatic with the aid of diboranes of the general formula

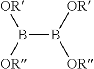

in which R′ and R″ are each as defined above, in the presence of an aprotic organic solvent, of a transition metal catalyst and of a base. Suitable diboranes are especially bis(1,2- and 1,3-diolato)diboranes, tetraalkoxydiboranes, tetracycloalkoxydiboranes and tetra(het)aryloxydiboranes, and mixed forms thereof. Examples of these compounds include: bis(pinacolato)diborane, bis(1,2-benzodiolato)diborane, bis(2,2-dimethyl-1,3-propanediolato)diborane, bis(1,1,3,3-tetramethyl-1,3-propanediolato)diborane, bis(4,5-pinanediolato)diborane, bis(tetramethoxy)diborane, bis(tetracyclopentoxy)diborane, bis(tetraphenoxy)diborane and bis(4-pyridiyloxy)diborane. Boronic acid derivatives VII can be prepared, for example, as described in WO 03/104232, WO 2006/111511, WO 2006/117383 or WO 2008/052927.

The halonaphthalene compounds can be prepared in analogy to known processes or as described in the examples. Haloperyleneimide compounds are known from WO 2004/029028.

The key step in process variant A is the cyclodehydrogenation in step ii). The cyclodehydrogenation can be effected by various methods. In a preferred process variant, the cyclodehydrogenation is effected in the presence of a Lewis acid and of an inert solvent. Suitable Lewis acids are, for example, iron(III) trihalides such as iron(III) chloride or iron(III) bromide, especially iron(III) chloride. Useful inert solvents include in particular polar aprotic organic solvents such as halogenated or nitrated hydrocarbons, cyclic ethers, aliphatic ethers or mixtures thereof. Examples of particularly suitable solvents are dichloromethane or nitromethane. In general, the amount of iron(III) trihalide is 2 to 15 mol, preferably 5 to 10 mol, per mole of the compound of the formula (IX).

Likewise suitable as Lewis acids are aluminum trihalides such as aluminum trichloride and aluminum tribromide, preference being given to aluminum trichloride. In the case of use of aluminum trihalide as a Lewis acid, all organic solvents which are inert under reaction conditions are useful in principle, preference being given to polar aprotic solvents. Examples of particularly suitable solvents are halogenated or nitrated aromatic hydrocarbons, such as chlorobenzene, di- and trichlorobenzenes and nitrobenzene. Typically 2 to 10 mol, preferably 6 to 8 mol, of aluminum trihalide are used per mole of the compound of the formula II. The reaction temperature is generally 20° C. up to the boiling temperature of the solvent, preferably 45 to 130° C. In a further variant, the cyclodehydrogenation is effected under Kovacic conditions with AlCl₃ in the presence of copper(II) chloride in CS₂ or with AlCl₃ in the presence of copper triflate in CS₂, as described in Angew. Chem. Int. Ed. Engl. 1995, 34, 1583.

In a further process variant, the cyclodehydrogenation is effected in a base-stable high-boiling organic solvent in the presence of an alkali metal or alkaline earth metal base and of an auxiliary nitrogen base. Suitable solvents are nonpolar aprotic, polar aprotic and protic solvents.

Preferred nonpolar aprotic solvents are xylenes, mesitylene, toluene and decalin; preferred polar aprotic solvents are diphenyl ether and the dialkyl ethers of monomeric and oligomeric ethylene glycol, especially diethylene glycol dimethyl and diethyl ether. In addition to the aprotic organic solvents, it is also possible to use protic solvents which comprise amino and hydroxyl functions. Suitable examples are alcoholamines, especially mono-, di- and tri-C₂-C₄-alcoholamines such as mono-, di- and triethanolamine, particular preference being given to ethanolamine.

Suitable bases are strong inorganic and organic alkali metal or alkaline earth metal bases, the alkali metal bases being particularly suitable. Preferred inorganic bases are alkali metal and alkaline earth metal hydroxides and amides; preferred organic bases are alkali metal and alkaline earth metal alkoxides, especially the C₁-C₁₀-alkoxides, in particular tert-C₄-C₆-alkoxides, alkali metal and alkaline earth metal (phenyl)alkylamides, especially the bis(C₁-C₄-alkyl)amides and triphenylmethyl metallates. Particular preference is given to the alkali metal alkoxides. Preferred alkali metals are lithium, sodium and potassium, very particular preference being given to potassium. Particularly suitable alkaline earth metals are magnesium and calcium. It is of course also possible to use mixtures of different bases.

Examples of particularly preferred bases include lithium hydroxide, sodium hydroxide and potassium hydroxide; lithium amide, sodium amide and potassium amide; lithium methoxide, sodium methoxide, potassium methoxide, lithium ethoxide, sodium ethoxide, potassium ethoxide, sodium isopropoxide, potassium isopropoxide, sodium tert-butoxide, potassium tert-butoxide, lithium (1,1-dimethyl)octoxide, sodium (1,1-dimethyl)octoxide and potassium (1,1-dimethyl)octoxide. Very particularly preferred bases are lithium diisopropylamide, sodium methoxide, sodium tert-butoxide, in particular potassium methoxide and potassium hydroxide, and especially sodium tert-butoxide and potassium tert-butoxide.

Especially in the case of use of the alkoxides and of the hydroxides, the reactivity is increased by adding an auxiliary nitrogen base with low nucleophilic action. Suitable bases are alkylamines liquid at the reaction temperatures, especially tri(C₃-C₆-alkyl)amines such as tripropylamine and tributylamine, alcoholamines, especially mono-, di- and tri(C₂-C₄-alcohol)amines such as mono-, di- and triethanolamine, and especially heterocyclic bases such as pyridine, N-methylpiperidine, N-methylpiperidone, N-methylmorpholine, N-methyl-2-pyrrolidone, pyrimidine, quinoline, isoquinoline, quinaldine, and in particular diazabicyclononene (DBN) and diazabicycloundecene (DBU). It will be appreciated that it is also possible to use mixtures of these auxiliary bases.

The reaction temperature is typically 50 to 210° C., preferably 20 to 170° C.

### Process Variant A2:

Process variant A2 is especially suitable for preparation of compounds I in which A is a VI.4 or VI.5 radical.

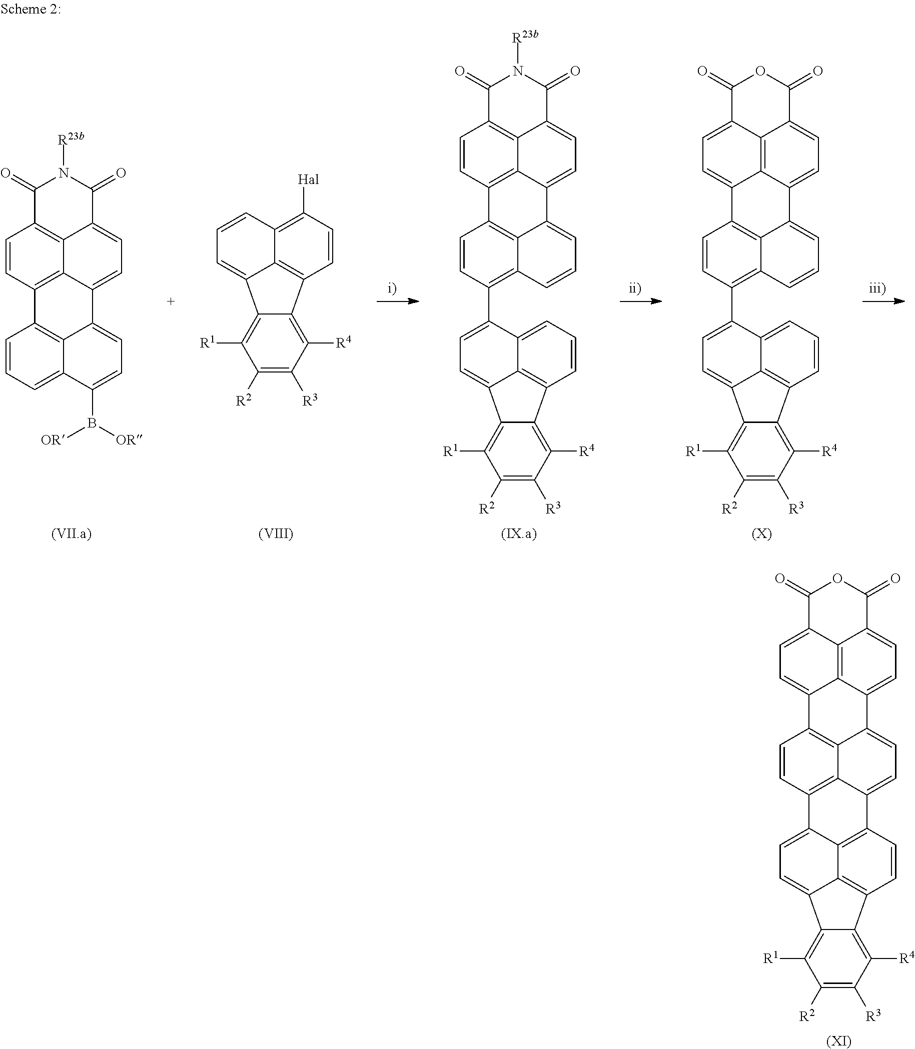

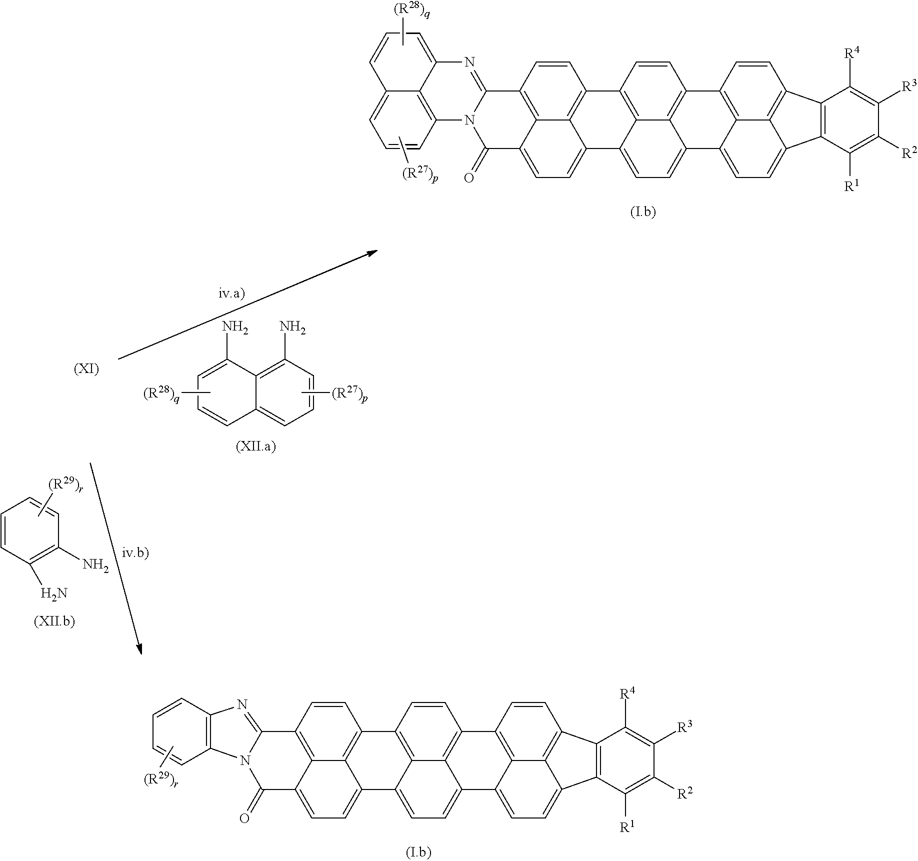

In scheme 2, p, q, r, R¹, R², R³, R⁴, R²⁷, R²⁸ and R²⁹ each have the definitions specified above, especially the definitions specified as preferred. R^{23b} is phenyl which is unsubstituted or substituted by 1 or 2 C₁-C₂₀-alkyl radicals. Hal is chlorine, bromine or iodine, preferably bromine. R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene which optionally bears 1, 2, 3, 4, 5, 6, 7 or 8 substituents selected from C₁-C₄-alkyl, C₅-C₈-cycloalkyl, C₆-C₁₄-aryl or hetaryl. Preferably, R′ and R″ are each independently hydrogen or C₁-C₄-alkyl or R′ and R″ together are C₂-C₄-alkylene optionally substituted by 1, 2, 3 or 4 C₁-C₄-alkyl groups.

In step i) of scheme 2, the perylenemonoimideboronic acid derivative VII.a is reacted with the halogen compound XIII to obtain the compound of the formula IX.a in analogy to step i) in scheme 1. The reaction product IX.a obtained in step i) is subsequently subjected to a hydrolysis. The hydrolysis of IX.a to X can be effected in analogy to known processes. The hydrolysis is typically effected in inert polar solvents such as C₁-C₆-alkanols, especially tert-butanol, preferably with inorganic bases such as alkali metal or alkaline earth metal hydroxides, especially NaOH or KOH.

Alternatively, the compound IX.a is also obtainable by reacting a halogen compound VII.b with a boronic acid derivative VIII.a as shown in scheme 2a. In scheme 2a, R^{23b}, R′, R″, R¹, R², R³ and R⁴ each have the definitions specified in scheme 2, especially the preferred definitions. The reaction is effected in analogy to step i) in scheme 1.

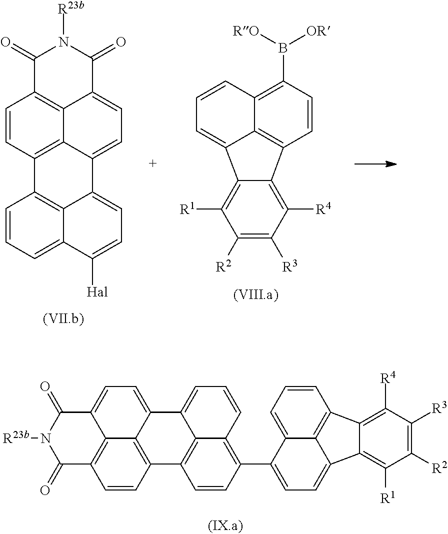

In step iii), the anhydride X thus obtained is subjected to a cyclodehydrogenation under the conditions described in step ii) in scheme 1 to obtain the compound Xl.

The imidation of carboxylic anhydride groups in the final step is known in principle, for example from WO 2007/116001. The reaction of the anhydride (XI) with the 1,8-diaminonaphthalene compound XII.a in step iv.a) gives rise to the compound I.b in which A is a radical of the formula VI.4. The reaction of the anhydride (XI) with 1,2-diaminobenzene XII.b in step iv.b) gives rise to the compound I.b in which A is a radical of the formula VI.5.

Preference is given to effecting the imidation in the presence of a polar aprotic solvent. Suitable polar aprotic solvents are nitrogen heterocycles such as pyridine, pyrimidine, quinoline, isoquinoline, quinaldine, N-methylpiperidine, N-methylpiperidone and N-methylpyrrolidone.

The imidation is effected preferably in the presence of an imidation catalyst. Suitable imidation catalysts are organic and inorganic acids, for example formic acid, acetic acid, propionic acid and phosphoric acid. Suitable imidation catalysts are additionally organic and inorganic salts of transition metals such as zinc, iron, copper and magnesium. Examples include zinc acetate, zinc propionate, zinc oxide, iron(II) acetate, iron(III) chloride, iron(II) sulfate, copper(II) acetate, copper(II) oxide and magnesium acetate. In general, the imidation catalyst and the diamino compound of the formula XII.a or XII.b are used in approximately stoichiometric amounts.

The reaction temperature is generally ambient temperature to boiling temperature of the solvent, preferably 40° C. to 180° C.

Preferably, the water of reaction which forms and any water introduced by the assistants are distilled off during the reaction.

A specific embodiment of the process shown in scheme 2 envisages performance of steps iii) and iv.a) or iv.b) in reverse sequence. In this embodiment, the compound of the formula X is first subjected to a reaction with the diamine XII.a or XII.b under the conditions described above. Subsequently, the imide obtained is subjected to a cyclodehydrogenation under the conditions described above.

The diamino compounds of the formula XII.a or XII.b are known from the literature or can be prepared by processes known from the literature.

Compounds of the formula I in which A is a VI.1 radical can also be prepared by the process shown in scheme 3.

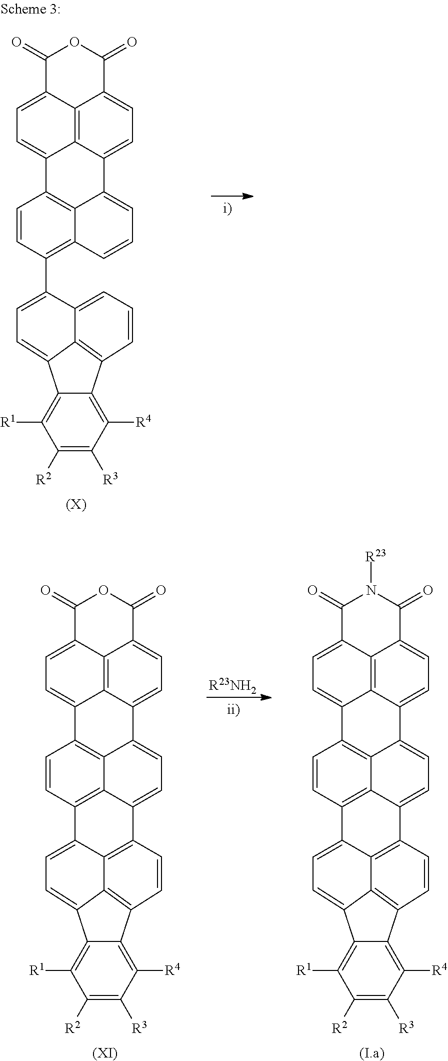

In scheme 3, R¹, R², R³, R⁴ and R²³ each have the definitions specified above, especially the definitions specified as preferred.

Step i) in scheme 3 can be performed under the conditions described in step ii) in scheme 2.

The reaction of the anhydride X with the primary amine R²³NH₂ is effected typically in the presence of a high-boiling solvent. The imidation in step ii) is effected under reaction conditions known per se, as known, for example, from WO 2007/116001.

A specific embodiment of the process shown in scheme 3 envisages performing steps i) and ii) in reverse sequence.

The process shown in scheme 3 is especially suitable for preparation of compound I.a in which R²³ is hydrogen.

### Process Variant B:

Process variant B by [1+1+1] synthesis from three naphthalene units comprises a sequence of coupling and cyclodehydrogenation, another coupling and cyclodehydrogenation, proceeding from a naphthaleneboronic acid derivative and a naphthalene compound which bears leaving groups suitable for a coupling reaction in the 1,4 or 1,5 positions. A procedure suitable for process technology purposes is shown in scheme 4.

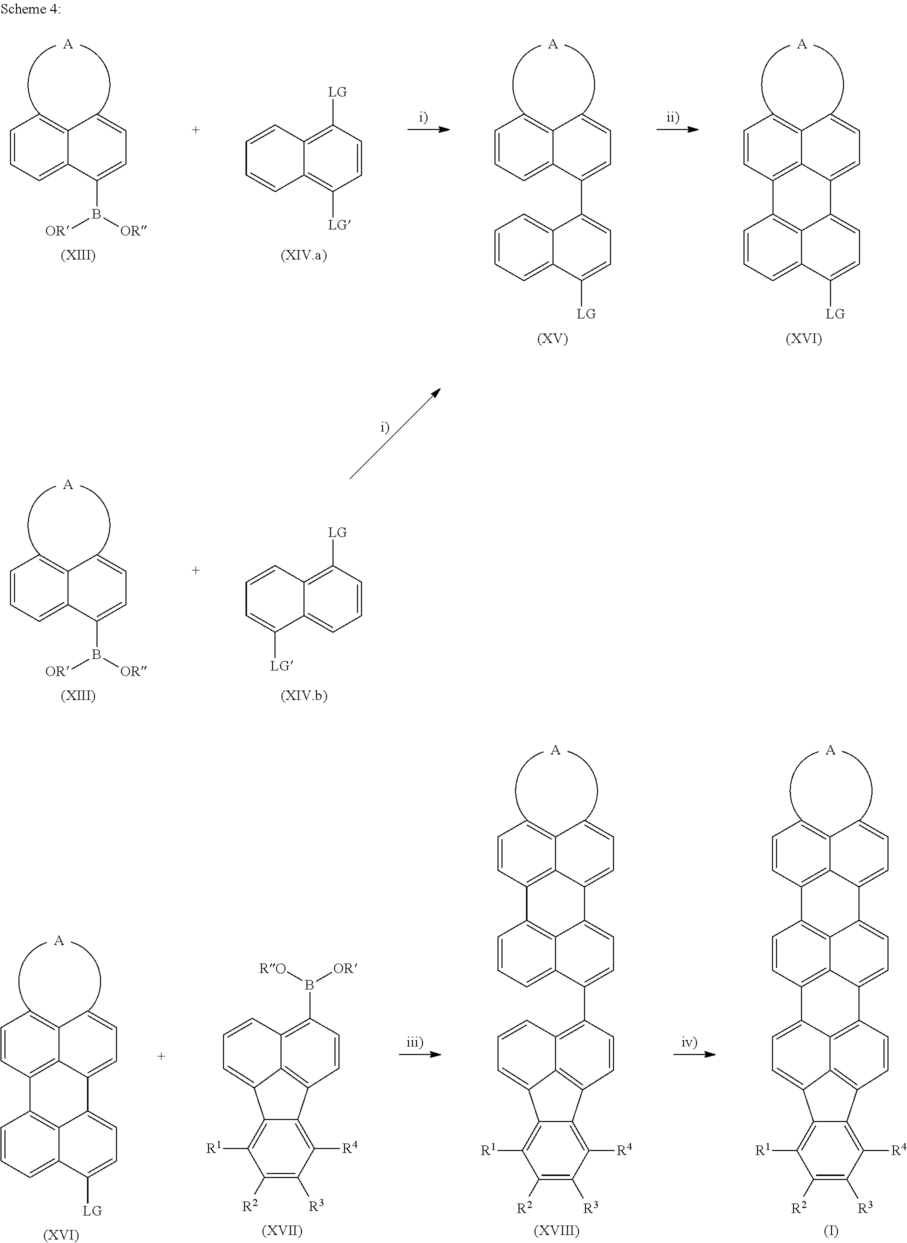

In scheme 4 R¹, R², R³ and R⁴ each have the definitions specified above, especially the definitions specified as preferred. LG is tosylate, chlorine, bromine or iodine, preferably bromine. LG′ is tosylate, chlorine, bromine or iodine, preferably bromine. A is a radical of the formula VI.1, VI.2, VI.3, VI.4, VI.5 or VI.6. R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene which optionally bears 1, 2, 3, 4, 5, 6, 7 or 8 substituents selected from C₁-C₄-alkyl, C₅-C₈-cycloalkyl, C₆-C₁₄-aryl and hetaryl. Preferably, R′ and R″ are each independently hydrogen or C₁-C₄-alkyl or R′ and R″ together are C₂-C₄-alkylene optionally substituted by 1, 2, 3 or 4 C₁-C₄-alkyl groups.

In step i) in scheme 4, the compound of the formula XIII is subjected to a coupling reaction with a naphthalene compound of the formula XIV.a or of the formula XIV.b. In the compound XIV, the LG and LG′ radicals may be the same or different; they are preferably the same. The coupling is effected preferably under the conditions described in step i) in scheme 1. The cyclodehydrogenation in step ii) is effected preferably under the conditions described in step ii) in scheme 1. Subsequently, in step iii), the monohalogen compound XVI is coupled with a naphthaleneboronic acid derivative of the formula XVII to obtain the compound XVIII. The subsequent cyclodehydrogenation in step iv) gives rise to the compound of the formula I. Steps iii) and iv) are preferably effected as described in steps i) and ii) in scheme 1.

1,4-Dibromonaphthalene, 1,5-dibromonaphthalene and 1,4-dichloronaphthalene are commercially available. Commercially unavailable dihalonaphthalene compounds can be prepared by processes known from the literature.

The compounds XIII are either known or can be prepared in analogy to the synthesis of the perylenemonoimideboronic acid derivatives VII. Compounds of the formula I in which A is a VI.3 radical are obtainable, for example, proceeding from commercially available 3-bromobenzanthrone.

### Organic Solar Cells

Before use in an organic solar cell, the terrylene compound can be subjected to purification. The purification can be effected by customary methods known to those skilled in the art, such as separation on suitable stationary phases, sublimation, extraction, distillation, recrystallization or a combination of at least two of these measures. Each purification may have a one-stage or multistage configuration. Individual purifying operations can be repeated twice or more. Different purifying operations can be combined with one another.

As described above, the inventive terrylene compounds of the general formula (I) and those used in the inventive solar cells can surprisingly be sublimed. In a specific embodiment, the purification therefore comprises a sublimation. This may preferably be a fractional sublimation. For fractional sublimation, it is possible to use a temperature gradient in the sublimation and/or the deposition of the terrylene compound. In addition, the purification can be effected by sublimation with the aid of a carrier gas stream. Suitable carrier gases are inert gases, for example nitrogen, argon or helium. The gas stream laden with the compound can subsequently be passed into a separating chamber. Suitable separating chambers may have a plurality of separation zones which can be operated at different temperatures. Preference is given, for example, to a so-called three-zone sublimation apparatus. A further process and an apparatus for fractional sublimation are described in U.S. Pat. No. 4,036,594.

In an alternative embodiment, the purification of the terrylene compounds of the general formula (I) comprises a column chromatography method. To this end, the starting material present in a solvent or solvent mixture can be subjected, for example, to a separation or filtration on silica gel. Finally, the solvent is removed, for example by evaporation under reduced pressure. Suitable solvents are aromatics such as benzene, toluene, xylene, mesitylene, chlorobenzene or dichlorobenzene, hydrocarbons and hydrocarbon mixtures, such as pentane, hexane, ligroin and petroleum ether, halogenated hydrocarbons such as chloroform or dichloromethane, and mixtures of the solvents mentioned. For chromatography, it is also possible to use a gradient of at least two different solvents, for example a toluene/petroleum ether gradient.

Organic solar cells generally have a layer structure and generally comprise at least the following layers: anode, photoactive layer and cathode. These layers are generally applied to a substrate suitable for this purpose. The structure of organic solar cells is described, for example, in US 2005/0098726 and US 2005/0224905.

The invention provides an organic solar cell which comprises a substrate with at least one cathode and at least one anode, and at least one terrylene compound of the general formula (I) as defined above as a photoactive material. The inventive organic solar cell comprises at least one photoactive region. A photoactive region may comprise two layers, each of which has a homogeneous composition and forms a flat donor-acceptor heterojunction. A photoactive region may also comprise a mixed layer and form a donor-acceptor heterojunction in the form of a donor-acceptor bulk heterojunction. Organic solar cells with photoactive donor-acceptor transitions in the form of a bulk heterojunction are a preferred embodiment of the invention.

Suitable substrates for organic solar cells are, for example, oxidic materials, polymers and combinations thereof. Preferred oxidic materials are selected from glass, ceramic, SiO₂, quartz, etc. Preferred polymers are selected from polyethylene terephthalates, polyolefins (such as polyethylene and polypropylene), polyesters, fluoropolymers, polyamides, polyurethanes, polyalkyl(meth)acrylates, polystyrenes, polyvinyl chlorides and mixtures and composites.

Suitable electrodes (cathode, anode) are in principle metals, semiconductors, metal alloys, semiconductor alloys, nanowire thereof and combinations thereof. Preferred metals are those of groups 2, 8, 9, 10, 11 or 13 of the periodic table, e.g. Pt, Au, Ag, Cu, Al, In, Mg or Ca. Preferred semiconductors are, for example, doped Si, doped Ge, indium tin oxide (ITO), fluorinated tin oxide (FTO), gallium indium tin oxide (GITO), zinc indium tin oxide (ZITO), poly(3,4-ethylenedioxythiophene) poly(styrenesulfonate) (PEDOT-PSS), etc. Preferred metal alloys are, for example, alloys based on Pt, Au, Ag, Cu, etc. A specific embodiment is Mg/Ag alloys.

The material used for the electrode facing the light (the anode in a normal structure, the cathode in an inverse structure) is preferably a material at least partly transparent to the incident light. This preferably includes electrodes which have glass and/or a transparent polymer as a carrier material. Transparent polymers suitable as carriers are those mentioned above, such as polyethylene terephthalate. The electrical contact connection is generally effected by means of metal layers and/or transparent conductive oxides (TCOs). These preferably include ITO, doped ITO, FTO (fluorine doped tin oxide), AZO (aluminum doped tin oxide), ZnO, TiO₂, Ag, Au, Pt. Particular preference is given to ITO for contact connection. For electrical contact connection, it is also possible to use a conductive polymer, for example a poly-3,4-alkylenedioxy-thiophene, e.g. poly-3,4-ethyleneoxythiophene poly(styrenesulfonate) (PEDOT).

The electrode facing the light is configured such that it is sufficiently thin to bring about only minimal light absorption but thick enough to enable good charge transport of the extracted charge carriers. The thickness of the electrode layer (without carrier material) is preferably within a range from 20 to 200 nm.

In a specific embodiment, the material used for the electrode facing away from the light (the cathode in a normal structure, the anode in an inverse structure) is a material which at least partly reflects the incident light. This includes metal films, preferably of Ag, Au, Al, Ca, Mg, In, and mixtures thereof. Preferred mixtures are Mg/Al. The thickness of the electrode layer is preferably within a range from 20 to 300 nm.

The photoactive region comprises or consists of at least one layer which comprises at least one terrylene compound of the general formula (I) as defined above. In addition, the photoactive region may have one or more further layer(s). These are, for example, selected from
layers with electron-conducting properties (electron transport layer, ETL),
layers which comprise a hole-conducting material (hole transport layer, HTL),
which need not absorb any radiation,
exciton- and hole-blocking layers (e.g. EBLs), which must not absorb, and
multiplication layers.

Suitable materials for these layers are described in detail hereinafter.

Suitable exciton- and hole-blocking layers are described, for example, in U.S. Pat. No. 6,451,415. Suitable materials for exciton-blocking layers are, for example, bathocuproin (BCP), 4,4′,4″-tris[3-methylphenyl-N-phenylamino]triphenylamine (m-MTDATA).

The inventive solar cells comprise at least one photoactive donor-acceptor heterojunction. Optical excitation of an organic material generates excitons. In order that a photocurrent occurs, the electron-hole pair has to be separated, typically at a donor-acceptor interface between two unlike contact materials. At such an interface, the donor material forms a heterojunction with an acceptor material. When the charges are not separated, they can recombine in a process also known as “quenching”, either radiatively by the emission of light of a lower energy than the incident light or nonradiatively by generation of heat. Both processes are undesired. According to the invention, at least one substituted terrylene of the general formula (I) can be used as a charge generator (donor). In combination with an appropriate electron acceptor material (ETM, electron transport material), radiative excitation is followed by a rapid electron transfer to the ETM. Suitable ETMs are, for example, C60 and other fullerenes, perylene-3,4; 9,10-bis(dicarboximides) (PTCDIs), or n-doped layers thereof (as described hereinafter). Preferred ETMs are C60 and other fullerenes or n-doped layers thereof.

In a first embodiment, the heterojunction has a flat configuration (see: Two layer organic photovoltaic cell, C. W. Tang, Appl. Phys. Lett., 48 (2), 183-185 (1986) or N. Karl, A. Bauer, J. Holzäpfel, J. Marktanner, M. Mobus, F. Stolzle, Mol. Cryst. Liq. Cryst., 252, 243-258 (1994).).

In a second preferred embodiment, the heterojunction is configured as a bulk (mixed) heterojunction, also referred to as an interpenetrating donor-acceptor network. Organic photovoltaic cells with a bulk heterojunction are described, for example, by C. J. Brabec, N. S. Sariciftci, J. C. Hummelen in Adv. Funct. Mater., 11 (1), 15 (2001) or by J. Xue, B. P. Rand, S. Uchida and S. R. Forrest in J. Appl. Phys. 98, 124903 (2005). Bulk heterojunctions are discussed in detail hereinafter.

The compounds of the formula (I) can be used as a photoactive material in cells with MiM, pin, pn, Mip or Min structure (M=metal, p=p-doped organic or inorganic semiconductor, n=n-doped organic or inorganic semiconductor, i=intrinsically conductive system of organic layers; see, for example, J. Drechsel et al., Org. Electron., 5 (4), 175 (2004) or Maennig et al., Appl. Phys. A 79, 1-14 (2004)).

The compounds of the formula (I) can also be used as a photoactive material in tandem cells. Suitable tandem cells are described, for example, by P. Peumans, A. Yakimov, S. R. Forrest in J. Appl. Phys., 93 (7), 3693-3723 (2003) (see also U.S. Pat. No. 4,461,922, U.S. Pat. No. 6,198,091 and U.S. Pat. No. 6,198,092) and are described in detail hereinafter.

The use of terrylene compounds of the general formula (I) in tandem cells is a preferred embodiment of the invention.

The compounds of the formula (I) can also be used as a photoactive material in tandem cells which are constructed from two or more than two stacked MiM, pin, Mip or Min structures (see DE 103 13 232.5 and J. Drechsel et al., Thin Solid Films, 451452, 515-517 (2004)).

The layer thickness of the M, n, i and p layers is typically within a range from 10 to 1000 nm, more preferably from 10 to 400 nm. The layers which form the solar cell can be produced by customary processes known to those skilled in the art. These include vapor deposition under reduced pressure or in an inert gas atmosphere, laser ablation or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

In order to improve the efficiency of organic solar cells, it is possible to shorten the mean distance through which the exciton has to diffuse in order to arrive at the next donor-acceptor interface. To this end, it is possible to use mixed layers of donor material and acceptor material which form an interpenetrating network in which internal donor-acceptor heterojunctions are possible. This bulk heterojunction is a specific form of the mixed layer, in which the excitons generated need only travel a very short distance before they arrive at a domain boundary, where they are separated.

In a preferred embodiment, the photoactive donor-acceptor transitions in the form of a bulk heterojunction are produced by a gas phase deposition process (physical vapor deposition, PVD). Suitable processes are described, for example, in US 2005/0227406, to which reference is made here. To this end, a terrylene compound of the general formula (I) and a complementary semiconductor material can be subjected to a gas phase deposition in the manner of a cosublimation. PVD processes are performed under high-vacuum conditions and comprise the following steps: evaporation, transport, deposition. The deposition is effected preferably at a pressure within a range from about 10⁻² mbar to 10⁻⁷ mbar, for example from 10⁻⁵ to 10⁻⁷ mbar. The deposition rate is preferably within a range from 0.01 to 100 nm/s. The deposition can be effected in an inert gas atmosphere, for example under nitrogen, helium or argon. The temperature of the substrate during the deposition is preferably within a range from −100 to 300° C., more preferably from −50 to 250° C.

The other layers of the organic solar cell can be produced by known processes. These include vapor deposition under reduced pressure or in an inert gas atmosphere, laser ablation, or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

The photoactive layer (homogeneous layer or mixed layer) can be subjected to a thermal treatment directly after production thereof or after production of further layers which form the solar cell. Such a heat treatment can in many cases further improve the morphology of the photoactive layer. The temperature is preferably within a range from about 60° C. to 300° C. The treatment time is preferably within a range from 1 minute to 3 hours. In addition or alternatively to a thermal treatment, the photoactive layer (mixed layer) can be subjected to a treatment with a solvent-containing gas directly after production thereof or after production of further layers which form the solar cell. In a suitable embodiment, saturated solvent vapors in air are used at ambient temperature. Suitable solvents are toluene, xylene, chloroform, N-methylpyrrolidone, dimethylformamide, ethyl acetate, chlorobenzene, dichloromethane and mixtures thereof. The treatment time is preferably within a range from 1 minute to 3 hours.

In a suitable embodiment, the inventive solar cells are present as an individual cell with flat heterojunction and normal structure. In a specific embodiment, the cell has the following structure:
an at least partly transparent conductive layer (top electrode, anode) (11)
a hole-conducting layer (hole transport layer, HTL) (12)
a layer which comprises a donor material (13)
a layer which comprises an acceptor material (14)
an exciton-blocking and/or electron-conducting layer (15)
a second conductive layer (back electrode, cathode) (16)

The donor material preferably comprises at least one compound of the formula (I) or consists of a compound of the formula (I). The acceptor material preferably comprises at least one fullerene or fullerene derivative, or consists of a fullerene or fullerene derivative. The acceptor material preferably comprises C60 or PCBM ([6,6]-phenyl-C61-butyric acid methyl ester).

The essentially transparent conductive layer (11) (anode) comprises a carrier, such as glass or a polymer (e.g. polyethylene terephthalate) and a conductive material, as described above. Examples include ITO, doped ITO, FTO, ZnO, AZO, etc. The anode material can be subjected to a surface treatment, for example with UV light, ozone, oxygen plasma, Br₂, etc. The layer (11) should be sufficiently thin to enable maximum light absorption, but also sufficiently thick to ensure good charge transport. The layer thickness of the transparent conductive layer (11) is preferably within a range from 20 to 200 nm.

Solar cells with normal structure optionally have a hole-conducting layer (HTL). This layer comprises at least one hole-conducting material (hole transport material, HTM). Layer (12) may be an individual layer of essentially homogeneous composition or may comprise two or more than two sublayers.

Hole-conducting materials (HTM) suitable for forming layers with hole-conducting properties (HTL) preferably comprise at least one material with high ionization energy. The ionization energy is preferably at least 5.0 eV, more preferably at least 5.5 eV. The materials may be organic or inorganic materials. Organic materials suitable for use in a layer with hole-conducting properties are preferably selected from poly(3,4-ethylenedioxythiophene) poly(styrenesulfonate) (PEDOT-PSS), Ir-DPBIC (tris-N,N′-diphenylbenzimidazol-2-ylideneiridium(III)), N,N′-diphenyl-N,N′-bis(3-methylphenyl)-1,1′-diphenyl-4,4′-diamine (α-NPD), 2,2′,7,7′-tetrakis(N,N-di-p-methoxyphenylamine)-9,9′-spirobifluorene (spiro-MeOTAD), etc. and mixtures thereof. The organic materials may, if desired, be doped with a p-dopant which has a LUMO within the same range as or lower than the HOMO of the hole-conducting material. Suitable dopants are, for example, 2,3,5,6-tetrafluoro-7,7,8,8-tetracyanoquinodimethane (F₄TCNQ), WO₃, MoO₃, etc. Inorganic materials suitable for use in a layer with hole-conducting properties are preferably selected from WO₃, MoO₃, etc.

If present, the thickness of the layers with hole-conducting properties is preferably within a range from 5 to 200 nm, more preferably 10 to 100 nm.

Layer (13) comprises at least one terrylene compound of the general formula (I). The thickness of the layer should be sufficient to absorb a maximum amount of light, but thin enough to enable effective dissipation of the charge. The thickness of the layer (13) is preferably within a range from 5 nm to 1 μm, more preferably from 5 to 100 nm.

Layer (14) comprises at least one acceptor material. The acceptor material preferably comprises at least one fullerene or fullerene derivative. Alternatively or additionally suitable acceptor materials are specified hereinafter. The thickness of the layer should be sufficient to absorb a maximum amount of light, but thin enough to enable effective dissipation of the charge. The thickness of the layer (14) is preferably within a range from 5 nm to 1 μm, more preferably from 5 to 80 nm.

Solar cells with normal structure optionally comprise an exciton-blocking and/or electron-conducting layer (15) (EBL/ETL). Suitable materials for exciton-blocking layers generally have a greater band gap than the materials of layer (13) and/or (14). They are firstly capable of reflecting excitons and secondly enable good electron transport through the layer. The materials for the layer (15) may comprise organic or inorganic materials. Suitable organic materials are preferably selected from 2,9-dimethyl-4,7-diphenyl-1,10-phenanthroline (BCP), 4,7-diphenyl-1,10-phenanthroline (Bphen), 1,3-bis[2-(2,2′-bipyridin-6-yl)-1,3,4-oxadiazo-5-yl]benzene (BPY-OXD), etc. The organic materials may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. Inorganic materials suitable for use in a layer with electron-conducting properties are preferably selected from ZnO, etc. If present, the thickness of the layer (15) is preferably within a range from 5 to 500 nm, more preferably 10 to 100 nm.

Layer 16 is the cathode and preferably comprises at least one compound with low work function, more preferably a metal such as Ag, Al, Mg, Ca, etc. The thickness of the layer (16) is preferably within a range from about 10 nm to 10 μm, e.g. 10 nm to 60 nm.

In a further suitable embodiment, the inventive solar cells are present as an individual cell with a flat heterojunction and inverse structure.

In a specific embodiment, the cell has the following structure:
an at least partly transparent conductive layer (cathode) (11)
an exciton-blocking and/or electron-conducting layer (12)
a layer which comprises an acceptor material (13)
a layer which comprises a donor material (14)
a hole-conducting layer (hole transport layer, HTL) (15)
a second conductive layer (back electrode, anode) (16)

With regard to suitable and preferred materials for the layers (11) to (16), reference is made to the above remarks regarding the corresponding layers in solar cells with normal structure.

In a further preferred embodiment, the inventive solar cells are present as an individual cell with normal structure and have a bulk heterojunction. In a specific embodiment, the cell has the following structure:
an at least partly transparent conductive layer (anode) (21)
a hole-conducting layer (hole transport layer, HTL) (22)
a mixed layer which comprises a donor material and an acceptor material, which form a donor-acceptor heterojunction in the form of a bulk heterojunction (23)
an electron-conducting layer (24)
an exciton-blocking and/or electron-conducting layer (25)
a second conductive layer (back electrode, cathode) (26)

The layer (23) comprises at least one terrylene compound of the general formula (I) as a photoactive material, especially as a donor material. The layer (23) additionally comprises preferably at least one fullerene or fullerene derivative as an acceptor material. The layer (23) comprises especially C60 or PCBM ([6,6]-phenyl-C61-butyric acid methyl ester) as an acceptor material.

With regard to layer (21), reference is made completely to the above remarks regarding layer (11).

With regard to layer (22), reference is made completely to the above remarks regarding layer (12).

Layer (23) is a mixed layer which comprises at least one terrylene compound of the general formula (I) as a donor material. In addition, layer (23) comprises at least one acceptor material. As described above, the layer (23) can be produced by coevaporation or by solution processing using customary solvents. The mixed layer comprises preferably 10 to 90% by weight, more preferably 20 to 80% by weight, of at least one compound of the general formula (I), based on the total weight of the mixed layer. The mixed layer comprises preferably 10 to 90% by weight, more preferably 20 to 80% by weight, of at least one acceptor material, based on the total weight of the mixed layer. The thickness of the layer (23) should be sufficient to absorb a maximum amount of light, but thin enough to enable effective dissipation of the charge. The thickness of the layer (23) is preferably within a range from 5 nm to 1 μm, more preferably from 5 to 200 nm, especially 5 to 80 nm.

Solar cells with a bulk heterojunction comprise an electron-conducting layer (24) (ETL). This layer comprises at least one electron transport material (ETM). Layer (24) may be a single layer of essentially homogeneous composition or may comprise two or more than two sublayers. Suitable materials for electron-conducting layers generally have a low work function or ionization energy. The ionization energy is preferably not more than 3.5 eV. Suitable organic materials are preferably selected from the aforementioned fullerenes and fullerene derivatives, 2,9-dimethyl-4,7-diphenyl-1,10-phenanthroline (BCP), 4,7-diphenyl-1,10-phenanthroline (Bphen), 1,3-bis[2-(2,2′-bipyridin-6-yl)-1,3,4-oxadiazo-5-yl]benzene (BPY-OXD), etc. The organic materials used in layer (24) may, if desired, be doped with an n-dopant which has a HOMO within the same range as or lower than the LUMO of the electron-conducting material.

Suitable dopants are, for example, Cs₂CO₃, Pyronin B (PyB), Rhodamine B, cobaltocenes, etc. The thickness of the layer (23) is, if present, preferably within a range from 1 nm to 1 μm, particularly 5 to 60 nm.

With regard to layer (25), reference is made completely to the above remarks regarding layer (15).

With regard to layer (26), reference is made completely to the above remarks regarding layer (16).

Solar cells with a donor-acceptor heterojunction in the form of a bulk heterojunction can be produced by a gas phase deposition process as described above. With regard to deposition rates, substrate temperature during the deposition and thermal aftertreatment, reference is made to the above remarks.

In a further preferred embodiment, the inventive solar cells are present as an individual cell with inverse structure and have a bulk heterojunction.

In a particularly preferred embodiment, the inventive solar cell is a tandem cell.

A tandem cell consists of two or more than two (e.g. 3, 4, 5, etc.) subcells. A single subcell, some of the subcells or all subcells may have photoactive donor-acceptor heterojunctions. Each donor-acceptor heterojunction may be in the form of a flat heterojunction or in the form of a bulk heterojunction. Preferably, at least one of the donor-acceptor heterojunctions is in the form of a bulk heterojunction. According to the invention, the photoactive layer of at least one subcell comprises a terrylene compound of the general formula (I). Preferably, the photoactive layer of at least one subcell comprises a terrylene compound of the general formula (I) and at least one fullerene or fullerene derivative. More preferably, the semiconductor mixture used in the photoactive layer of at least one subcell consists of a terrylene compound of the general formula (I) and C₆₀ or [6,6]-phenyl-C61-butyric acid methyl ester.

The subcells which form the tandem cell may be connected in parallel or in series. The subcells which form the tandem cell are preferably connected in series. There is preferably an additional recombination layer in each case between the individual subcells. The individual subcells have the same polarity, i.e. generally either only cells with normal structure or only cells with inverse structure are combined with one another.

FIG. 2 shows the basic structure of an inventive tandem cell. Layer 31 is a transparent conductive layer. Suitable materials are those specified above for the individual cells.

Layers 32 and 34 constitute subcells. “Subcell” refers here to a cell as defined above without cathode and anode. The subcells may, for example, either all have a terrylene compound of the general formula (I) used in accordance with the invention in the photoactive layer (preferably in combination with a fullerene or fullerene derivative, especially C60) or have other combinations of semiconductor materials, for example C60 with zinc phthalocyanine, C60 with oligothiophene (such as DCV5T). In addition, individual subcells may also be configured as dye-sensitized solar cells or polymer cells.

In all cases, preference is given to a combination of materials which exploit different regions of the spectrum of the incident light, for example of natural sunlight. For instance, the combination of terrylene compound of the general formula (I) and fullerene or fullerene derivative used in accordance with the invention absorbs in the long-wave region of sunlight. Cells based on at least one perylene compound as described, for example, in European patent application 10166498.5, absorb primarily in the short-wave range. Thus, a tandem cell composed of a combination of these subcells should absorb radiation in the range from about 400 nm to 900 nm. Suitable combination of subcells should thus allow the spectral range utilized to be extended. For optimal performance properties, optical interference should be considered. For instance, subcells which absorb at relatively short wavelengths should be arranged closer to the metal top contact than subcells with longer-wave absorption.

With regard to layer (31), reference is made completely to the above remarks regarding layers (11) and (21).

With regard to layers (32) and (34), reference is made completely to the above remarks regarding layers (12) to (15) for flat heterojunctions and (22) to (25) for bulk heterojunctions.

Layer 33 is a recombination layer. Recombination layers enable the charge carriers from one subcell to recombine with those of an adjacent subcell. Small metal clusters are suitable, such as Ag, Au or combinations of highly n- and p-doped layers. In the case of metal clusters, the layer thickness is preferably within a range from 0.5 to 5 nm. In the case of highly n- and p-doped layers, the layer thickness is preferably within a range from 5 to 40 nm. The recombination layer generally connects the electron-conducting layer of a subcell to the hole-conducting layer of an adjacent subcell. In this way, further cells can be combined to form the tandem cell.

Layer 36 is the top electrode. The material depends on the polarity of the subcells. For subcells with normal structure, preference is given to using metals with a low work function, such as Ag, Al, Mg, Ca, etc. For subcells with inverse structure, preference is given to using metals with a high work function, such as Au or Pt, or PEDOT-PSS.

In the case of subcells connected in series, the overall voltage corresponds to the sum of the individual voltages of all subcells. The overall current, in contrast, is limited by the lowest current of one subcell. For this reason, the thickness of each subcell should be optimized such that all subcells have essentially the same current.

Examples of different kinds of donor-acceptor heterojunctions are a donor-acceptor double layer with a flat heterojunction, or the heterojunction is configured as a hybrid planar-mixed heterojunction or gradient bulk heterojunction or annealed bulk heterojunction.

The production of a hybrid planar-mixed heterojunction is described in Adv. Mater. 17, 66-70 (2005). In this structure, mixed heterojunction layers which were formed by simultaneous evaporation of acceptor and donor material are present between homogeneous donor and acceptor material.

In a specific embodiment of the present invention, the donor-acceptor-heterojunction is in the form of a gradient bulk heterojunction. In the mixed layers composed of donor and acceptor materials, the donor-acceptor ratio changes gradually. The form of the gradient may be stepwise or linear. In the case of a stepwise gradient, the layer 01 consists, for example, of 100% donor material, layer 02 has a donor/acceptor ratio>1, layer 03 has a donor/acceptor ratio=1, layer 04 has a donor/acceptor ratio<1, and layer 05 consists of 100% acceptor material. In the case of a linear gradient, layer 01 consists, for example, of 100% donor material, layer 02 has a decreasing ratio of donor/acceptor, i.e. the proportion of donor material decreases in a linear manner in the direction of layer 03, and layer 03 consists of 100% acceptor material. The different donor-acceptor ratios can be controlled by means of the deposition rate of each and every material. Such structures can promote the percolation path for charges.

In a further specific embodiment of the present invention, the donor-acceptor heterojunction is configured as an annealed bulk heterojunction; see, for example, Nature 425, 158-162, 2003. The process for producing such a solar cell comprises an annealing step before or after the metal deposition. As a result of the annealing, donor and acceptor materials can separate, which leads to more extended percolation paths.

In a further specific embodiment of the present invention, the organic solar cells are produced by organic vapor phase deposition, either with a flat or a controlled heterojunction architecture. Solar cells of this type are described in Materials, 4, 2005, 37.

The inventive organic solar cells preferably comprise at least one photoactive region which comprises at least one terrylene compound of the formula (I) as a donor, which is in contact with at least one acceptor. Preferred acceptors are fullerenes and fullerene derivatives, preferably selected from C₆₀, C₇₀, C₈₄, phenyl-C₆₁-butyric acid methyl ester ([60]PCBM), phenyl-C₇₁-butyric acid methyl ester ([71]PCBM), phenyl-C₈₄-butyric acid methyl ester ([84]PCBM), phenyl-C₆₁-butyric acid butyl ester ([60]PCBB), phenyl-C₆₁-butyric acid octyl ester ([60]PCBO), thienyl-C₆₁-butyric acid methyl ester ([60]ThCBM) and mixtures thereof. Particular preference is given to C₆₀, [60]PCBM and mixtures thereof. Preference is given to those fullerenes which are vaporizable, for example C60 or C70.

In addition to terrylene compounds of the formula (I) and fullerenes, the semiconductor materials listed hereinafter are suitable in principle for use in the inventive solar cells. They serve especially as short-wave donors for subcells of a tandem cell, which are combined with a perylene/fullerene subcell used in accordance with the invention.

In a preferred embodiment, the tandem cell comprises a subcell comprising a terrylene compound of the general formula (I) and at least one fullerene or fullerene derivative, and additionally at least one subcell whose absorption maximum is in the relatively short-wave spectral range from 400 to 650 nm.

Suitable donors for the subcell whose absorption maximum is in the relatively short-wave spectral range from 400 to 650 nm are described hereinafter.

Suitable further semiconductors suitable predominantly as donors are perylenes of the formula

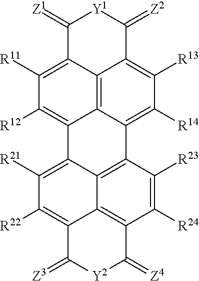

in which
the R¹¹, R¹², R¹³, R¹⁴, R²¹R²², R²³ and R²⁴ radicals are each independently hydrogen, halogen or groups other than halogen,
Y¹ is O or NR^{a} where R^{a} is hydrogen or an organyl radical,
Y² is O or NR^{b} where R^{b} is hydrogen or an organyl radical,
Z¹, Z², Z³ and Z⁴ are each 0,
where, in the case that Y¹ is NR^{a}, one of the Z¹ and Z² radicals may also be NR^{c}, where the R^{a} and R^{c} radicals together are a bridging group having 2 to 5 atoms between the flanking bonds, and
where, in the case that Y² is NR^{b}, one of the Z³ and Z⁴ radicals may also be NR^{d}, where the R^{b} and R^{d} radicals together are a bridging group having 2 to 5 atoms between the flanking bonds.

Suitable perylenes are, for example, described in WO 2007/074137, WO 2007/093643 and WO 2007/116001, to which reference is made here.

Semiconductors suitable as donors are thiophene compounds. These are preferably selected from thiophenes, oligothiophenes and substituted derivatives thereof. Suitable oligothiophenes are quaterthiophenes, quinquethiophenes, sexithiophenes, α,ω-di(C₁-C₈)-alkyloligothiophenes, such as α,ω-dihexylquaterthiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially am-alkyl-substituted phenylene-thiophene oligomers.

Further thiophene compounds suitable as semiconductors are preferably selected from compounds like

α,α′-bis(2,2-dicyanovinyl)quinquethiophene (DCV5T),
(3-(4-octylphenyl)-2,2′-bithiophene) (PTOPT),
and acceptor-substituted oligothiophenes as described in WO 2006/092124.

Further semiconductors suitable as donors are merocyanines as described in WO 2010/049512.

The inventive solar cell is more preferably a tandem cell. In that case, one subcell preferably has a photoactive region which comprises at least one terrylene compound of the formula (I) and C60.

All aforementioned semiconductors may be doped. The conductivity of semiconductors can be increased by chemical doping techniques using dopants. An organic semiconductor material may be doped with an n-dopant which has a HOMO energy level which is close to or higher than the LUMO energy level of the electron-conducting material. An organic semiconductor material may also be doped with a p-dopant which has a LUMO energy level which is close to or higher than the HOMO energy level of the hole-conducting material. In other words, in the case of n-doping an electron is released from the dopant, which acts as the donor, whereas in the case of p-doping the dopant acts as an acceptor which accepts an electron.

Suitable dopants for the terrylene compounds used in accordance with the invention and for p-semiconductors in general are, for example, selected from WO₃, MoO₃, 2,3,5,6-tetrafluoro-7,7,8,8-tetracyanoquinodimethane (F₄-TCNQ), 3,6-difluoro-2,5,7,7,8,8-hexacyanoquinodimethane, dichlorodicyanoquinone (DDQ) or tetracyanoquinodimethane (TCNQ). A preferred dopant is 3,6-difluoro-2,5,7,7,8,8-hexacyanoquinodimethane.

Suitable dopants for the p-semiconductors used in accordance with the invention are, for example, selected from Cs₂CO₃, LiF, Pyronin B (PyB), rhodamine derivatives, cobaltocenes, etc. Preferred dopants are Pyronin B and rhodamine derivatives, especially rhodamine B.

The dopants are typically used in an amount of up to 10 mol %, preferably up to 5 mol %, based on the amount of the semiconductor to be doped.

The invention is illustrated in detail with reference to the nonlimiting examples which follow.

### EXAMPLES

### I. Preparation of Precursors

### Example I.a

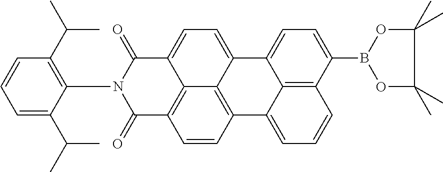

A suspension of 5.0 g (9 mmol) of 2,6-diisopropylphenylperylene-3,4-dicarboxylic acid monoimide (prepared as described in WO 2004/029028), 2.82 g (11.2 mmol) of bis(pinacolato)diboron, 0.3 g (0.18 mmol) of 1,1′-bis(diphenylphosphino)ferrocene-dichloropalladium, 0.98 g of potassium acetate (10 mmol) and 200 ml of toluene was heated while stirring under reflux under nitrogen for 16 hours. The reaction mixture was purified by chromatography on silica gel with 3:1 dichloromethane:toluene. This gave 2.0 g (37%) of the title compound as a red solid.

### Example I.b

### 7,9-Diphenyl-6b,7-dihydrocyclopenta[a]acenaphthylen-8-one

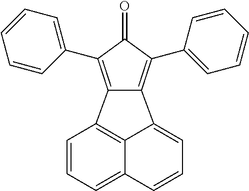

A suspension of 16.2 g (77 mmol) of 1,3-diphenylpropan-2-one and 14.2 g of acenaphthoquinone in 8.2 ml of toluene and 82 ml of ethanol was heated under reflux (78° C.). Subsequently, a solution of 1.35 g of KOH and 27 ml of ethanol was added dropwise within 15 minutes. The reaction mixture was stirred under reflux for a further 1 h. The reaction mixture was cooled to 0 to 5° C. The precipitated black product was filtered off with suction and washed with 3×100 ml of ethanol. This gave 26.2 g (yield: 96%) of the title compound. R_{f} (in 10:1 toluene:ethyl acetate): 0.8.

### Example I.c

### 7,11-Diphenyl-9-propyl-9-azacyclopenta[k]fluoranthene-8,10-dione

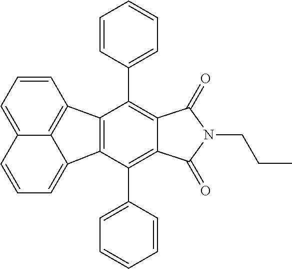

A mixture of 5.0 g (14 mmol) of 7,9-diphenyl-6b,7-dihydrocyclopenta[a]acenaphthylen-8-one from example I.b, 2.93 g (21.5 mmol) of n-propylmaleimide and 70 ml of chlorobenzene was heated under reflux (132° C.) for 3 hours. Subsequently, the reaction mixture was cooled to 70° C. and 2.9 g (18.25 mmol) of potassium permanganate and 4.9 g (18.25 mmol) of 18-crown-6 were added. The mixture was heated under reflux while stirring for a further 2.5 hours. The solution was filtered while still hot and the residue was washed with 3×10 ml of chlorobenzene. The precipitate which precipitates out of the filtrate at room temperature overnight was filtered off with suction. This gave 3.1 g (47.5% yield) of the title compound as a yellowish substance.

R_{f} (10:1 toluene:petroleum ether)=0.11.

### Example I.d

### 3-Bromo-7,11-diphenyl-9-propyl-9-azacyclopenta[k]fluoranthene-8,10-dione

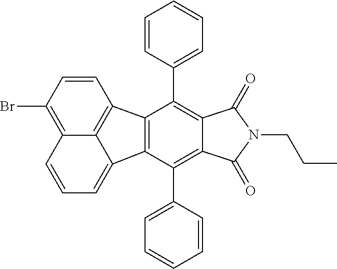

Under nitrogen, 5.5 ml (110 mmol) of bromine were added dropwise to a suspension of 2.5 g (5.37 mmol) of 7,11-diphenyl-9-propyl-9-azacyclopenta[k]fluoranthene-8,10-dione from Example I.c, a little iodine and 150 ml of glacial acetic acid. The mixture was stirred at 30° C. for 20 hours. Subsequently, the excess bromine was removed by introducing N2. The reaction mixture was stirred with 500 ml of 1% sodium thiosulfate solution for 30 min. The precipitate formed was filtered off with suction and washed with water. After recrystallization from toluene:petroleum ether, 3.1 g (100% yield) of the title compound were obtained as a yellow substance. R_{f} (dichloromethane): 0.42.

### Example I.e

### 3-Pinacolatoborylo-7,11-diphenyl-9-propyl-9-azacyclopenta[k]fluoranthene-8,10-dione

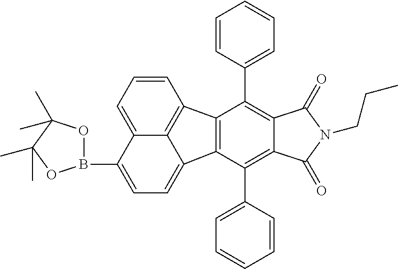

A mixture of 3.0 g (5.51 mmol) of 3-bromo-7,11-diphenyl-9-propyl-9-azacyclo-penta[k]fluoranthene-8,10-dione from example I.d, 4.0 g (16.5 mmol) of bis(pinacolato)diboron, 0.41 g (0.55 mmol) of 1,1′-bis(diphenylphosphino)ferrocenedichloropalladium, 1.62 g (43 mmol) of potassium acetate and 70 ml of toluene was stirred at 70° C. under nitrogen for 20 hours. The solvent was removed under reduced pressure. The crude product was purified by chromatography using silica gel (eluent: dichloromethane) and recrystallization in toluene/petroleum ether. This gave 2.5 g (76%) of the title compound as a yellow substance. R_{f} (dichloromethane)=0.34.

### Example I.f

### N-Phenyl-4-bromonaphthalene-1,8-dicarboxylic monoimide

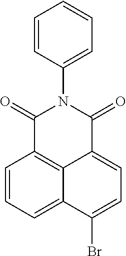

A mixture of 10.0 g (34 mmol) of 4-bromo-1,8-naphthalenedicarboxylic monoanhydride, 4.4 g (5.7 mmol) of aniline and 100 ml of propionic acid was heated to reflux overnight. After cooling to room temperature, the precipitate was filtered off with suction, washed with water and dried. This gave 10.1 g (68%) of the title compound as a colorless compound.

### Example I.g

### N-Phenyl-4-(pinacolatoboron)naphthalene-1,8-dicarboxylic monoimide

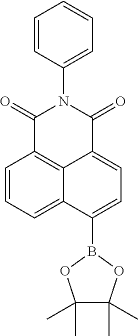

A mixture of 3.0 g (8.5 mmol) of the compound from example I.f, 3.2 g (12.8 mmol) of bispinacolatodiborane, 624 mg (0.8 mmol) of (1,1-bis(diphenyl-phosphino)ferrocene)dichloropalladium, 2.5 g (25.6 mmol) of potassium acetate in 100 ml of toluene was heated to 70° C. overnight. The salts were filtered off and, after removing the solvent, the filtrate was purified by chromatography with 5:1 cyclohexane/ethyl acetate. This gave 2.5 g (73%) of the title compound as a beige solid.

### Example I.h

### N-(1′-Heptyloctyl)-4-bromonaphthalene-1,8-dicarboxylic monoimide

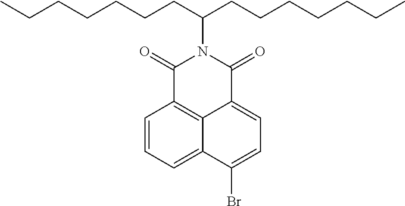

A mixture of 10.0 g (36 mmol) of 4-bromonaphthalenedicarboxylic monoimide, 14.0 g (43.3 mmol) of 1-heptyloctylamine and 5.3 g (29 mmol) of zinc acetate in 250 ml of quinoline was heated at reflux for 5 hours. After cooling to room temperature, the reaction mixture was poured onto 200 ml of 1M hydrochloric acid and then extracted with dichloromethane. The organic solvent was removed and the crude product was purified on silica gel using toluene. This gave 16.4 g (93%) of an oily product.

R_{f} (1:1 toluene:petroleum ether)=0.55.

### Example I.i

### N-(1′-Heptyloctyl)-4-(pinacolatoboron)naphthalene-1,8-dicarboxylic monoimide

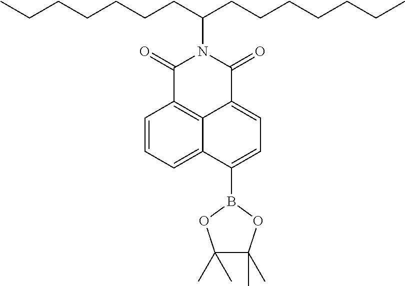

A mixture of 8.0 g (16.5 mmol) of the compound from example 1.e, 6.26 g (25 mmol) of bispinacolatodiborane, 1.2 g (1.6 mmol) of (1,1′-bis(diphenylphosphino)ferrocene)di-chloropalladium and 4.8 g (49 mmol) of potassium acetate in 200 ml of toluene was heated to 70° C. for 16 hours. After cooling to room temperature, insoluble constituents were filtered off and the product was purified on silica gel with 1:2 toluene/petroleum ether. This gave 6.4 g (73%) of a yellowish oil. R_{f} (1:1 toluene:petroleum ether)=0.16.

### Example I.k

### 3-Bromobenzo[de]benzo[4,5]imidazo[2,1-a]isoquinolin-7-one

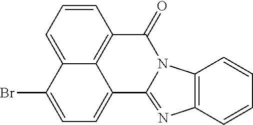

A mixture of 10.0 g (36 mmol) of 4-bromo-1,8-naphthalic anhydride, 4.68 g (43.3 mmol) of 1,2-phenylenediamine, 6.62 g of zinc acetate (36 mmol) and 100 ml of quinoline was heated at reflux at 145° C. while stirring for 5 hours. The product was then stirred into 500 ml of 1 molar hydrochloric acid. The precipitate was filtered off with suction, washed with hot water and recrystallized in toluene. This gave 10.6 g (84%) of the title compound as a yellow substance. R_{f} (2:1 cyclohexane:ethyl acetate)=0.29.

### Example I.l

### 3-Pinacolatoborylobenzo[de]benzo[4,5]imidazo[2,1-a]isoquinolin-7-one

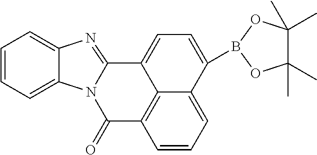

A mixture of 5.0 g (14.3 mmol) of 3-bromobenzo[de]benzo[4,5]imidazo[2,1-a]iso-quinolin-7-one from example I.k, 10.9 g (43 mmol) of bis(pinacolato)diboron, 1.05 g (1.43 mmol) of (1,1′-bis(diphenylphosphino)ferrocene)dichloropalladium, 4.22 g of potassium acetate (43 mmol) and 180 ml of toluene was stirred at 70° C. under nitrogen for 20 hours. The solvent was removed under reduced pressure. The crude product was purified by chromatography using silica gel (dichloromethane eluent) and recrystallization in toluene/petroleum ether. This gave 4.8 g (85%) of the title compound as a yellow substance. Rf (dichloromethane)=0.35.

### Example I.m

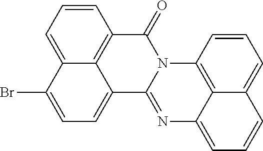

A mixture of 3.0 g (10.8 mmol) of 4-bromo-1,8-naphthalic anhydride, 2.1 g (13.0 mmol) of 1,8-diaminonaphthalene, 1.99 g of zinc acetate (10.8 mmol) and 300 ml of quinoline was heated at reflux at 145° C. while stirring for 5 hours. The product was stirred into 500 ml of 1 molar hydrochloric acid. The precipitate was filtered off with suction, washed with hot water and recrystallized in toluene. This gave 3.2 g (74%) of the title compound as a violet-red substance.

R_{f} (dichloromethane)=0.80.

### Example I.n

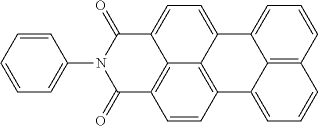

A mixture of 20.0 ml of quinoline, 1.0 g (2.5 mmol) of perylene-3,4-dicarboxylic anhydride (prepared as described in EP 0637436), 0.46 g (5.0 mmol) of aniline and 1.37 g (2.5 mmol) of zinc acetate was stirred at 100° C. for 16 hours. After cooling, the reaction mixture was stirred with 100 ml of 32% hydrochloric acid for 1 hour, and the precipitating product was filtered off with suction, washed to neutrality with hot water and dried. This gave 1.05 g (90.5% yield) of an orange compound. Rf (10:1 toluene/ethyl acetate)=0.4

### Example I.o

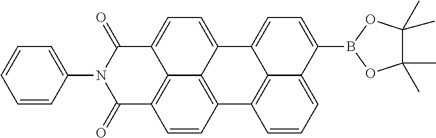

A suspension of 1.0 g (2.1 mmol) of the compound from example I.n, 1.53 g (6.0 mmol) of bis(pinacolato)diboron, 0.09 g (0.122 mmol) of (1,1-bis(diphenylphosphino)-ferrocene)palladium(II) chloride, 5.0 g of potassium acetate (5.2 mmol) and 20 ml of N-methylpyrrolidone was stirred at 70° C. under nitrogen for 16 hours. The mixture was poured onto water and the precipitating product was filtered off, washed with hot water and dried. This gave 1.09 g (99.1%) of a red substance.

R_{f} (dichloromethane)=0.41.

### Example I.p

### 3-Bromo-10-cyano-7,14-diphenylacenaptho[1,2-k]fluoranthene

### 4-Bromo-10-cyano-7,14-diphenylacenaptho[1,2-k]fluoranthene

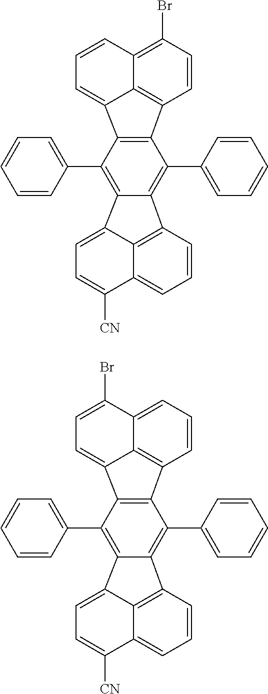

The compounds can be prepared as described in WO 2011/00939.

### Example I.1

### 3-Bromo-7,14-diphenylbenzofluoranthene

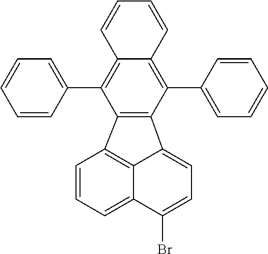

A mixture of 50 ml of N-methylpyrrolidone (NMP), 500 ml of dichloromethane, 10.0 g (25 mmol) of 7,14-diphenylbenzofluoranthene (prepared as described in WO 2010/031833) and 6.45 g (36 mmol) of N-bromosuccinimide was stirred at room temperature for 20 hours. Subsequently, the dichloromethane was removed under reduced pressure and the product was precipitated by adding water, filtered off and dried. This gave 12.1 g (quant.) of the title compound as a beige solid.

R_{f} (2:1 petroleum ether: toluene)=0.9.

### Example I.2

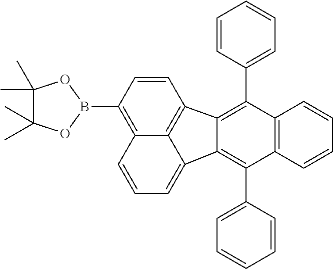

A suspension of 9.65 g (20 mmol) of the compound from example I.1, 10.2 g (40 mmol) of bis(pinacolato)diboron, 0.6 g (0.82 mmol) of 1,1′-bis(diphenylphosphino)-ferrocenedichloropalladium, 5.0 g of potassium acetate (52 mmol) and 200 ml of N-methylpyrrolidone was stirred at 70° C. under nitrogen for 16 hours. The mixture was poured onto water, and the precipitating product was filtered off, washed with hot water and dried. The crude product was chromatographed on silica gel with 3:1 petroleum ether:toluene. This gave 7.2 g (68%) of an orange substance. R_{f} (2:1 petroleum ether:toluene)=0.35.

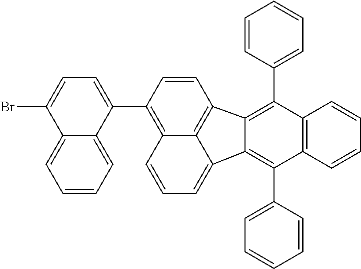

### Example I.3

2.15 g (7.5 mmol) of 1,4-dibromonaphthalene and 0.95 g (1.8 mmol) of the boron compound from example I.2 were dissolved in 40 ml of toluene at 40° C. Thereafter, a solution of 3.0 g of potassium carbonate, 20 ml of water and 8 ml of ethanol, and 0.5 g (0.43 mmol) of tetrakistriphenylphosphinepalladium(0) were added. The mixture was stirred at room temperature for 2 h. It was extracted with dichloromethane and purified by chromatography on silica gel with 10:1 petroleum ether:toluene. This gave 0.61 g (56%) of the title compound as a red compound. R_{f} (1:9 toluene:petroleum ether)=0.56.

### Example I.4

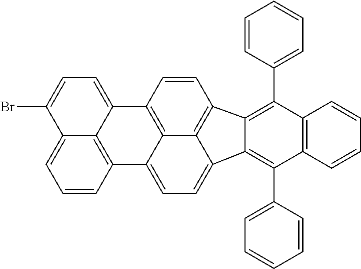

A mixture of 35 ml of argon-saturated chlorobenzene, 0.2 g (0.33 mmol) of the compound from example 1.3 and 1.0 g (13 mmol) of aluminum trichloride was stirred at 30° C. for 2.5 hours. Thereafter, the reaction mixture was extracted with water and the organic phase was concentrated under reduced pressure. The product was chromatographed using a silica gel-filled suction filter with 9:1 petroleum ether:toluene as the eluent. This gave 117 mg (59%) of the title compound as a red substance. R_{f} (dichloromethane)=0.53.

### Example I.5

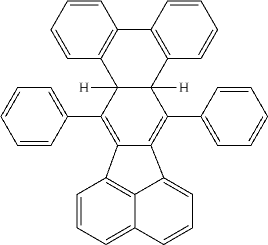

A solution of 15 g (40 mmol) of phencyclone and 7.6 g (40 mmol) of acenaphthylene in 300 ml of xylene was heated at reflux overnight. After cooling, the title compound was precipitated by adding ethanol. This gave 20.6 g (quant.) of a white solid.

R_{f} (5:1 toluene:petroleum ether)=0.5.

### Example I.6

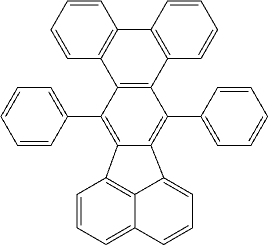

A mixture of 4.5 g (8.9 mmol) of the compound from example I.5, 13.0 g (57 mmol) of 2,3-dichloro-5,6-dicyanobenzoquinone (DDQ) in 1500 ml of chlorobenzene was heated to 134° C. for 60 hours. A solution of sodium thiosulfate was added, the chlorobenzene was distilled off and dichloromethane was added, which gave a solid. The chromatographic purification of the crude product on silica gel with 10:1 petroleum ether/toluene gave 1.4 g (31%) of the title compound.

R_{f} (1:5 toluene:petroleum ether)=0.32.

### Example I.7

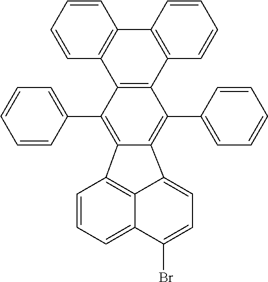

A mixture of 100 mg (0.2 mmol) of the compound from example I.6, 0.3 g (2 mmol) of bromine and 5 ml of glacial acetic acid was stirred at room temperature for 2 hours. Subsequently, the bromine was blown out and the product was isolated by filtration.

R_{f} (1:5 toluene:petroleum ether)=0.59.

### Example I.8

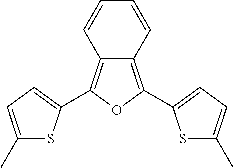

4.92 g (30 mmol) of 3-methoxy-3H-isobenzofuran-1-one in 50 ml of diethyl ether were added dropwise at −5° C. while stirring under a protective gas atmosphere to 63 ml of a 1 molar solution of 2-methylthiophenylmagnesium bromide in diethyl ether within 1 hour, and the suspension formed was stirred at this temperature for a further hour and then at room temperature overnight. The suspension was cooled to −5° C. and ice-water was added to the product. Then 1 molar HCl was added. The solution was extracted with dichloromethane and the organic phase was dried with sodium sulfate. The product was converted further in solution in the next step without further purification. R_{f} (10:1 toluene:ethyl acetate)=0.82.

### Example I.9

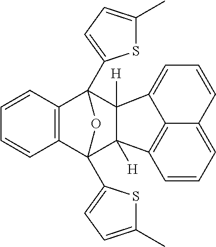

9.1 g (29.33 mmol) of the compound from Example I.8 in 700 ml of dichloromethane and diethyl ether were added dropwise at 100° C. to a solution of 4.46 g (29.3 mmol) of acenaphthylene in 150 ml of o-xylene. The low boilers were removed by means of a water separator. The mixture was stirred at reflux (140° C.) for 2 hours. The product was poured slowly onto 1 l of petroleum ether and stirred for a further 2 hours. The precipitate formed was filtered off and the filtrate was concentrated. This gave 16.33 g of the title compound (quantitative yield). R_{f} (100:1 toluene/acetone)=0.53.

### Example I.10

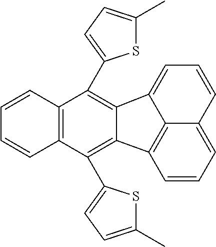

14.83 g (88 mmol) of 48% HBr were added to a solution of 13.55 g (29.3 mmol) of the compound from Example I.9 in 100 ml of acetic acid. The mixture was heated at reflux (approx. 110° C.) while stirring for 4 hours. The reaction mixture was poured onto water and the product was extracted with dichloromethane and toluene. The organic phase was dried and concentrated. This gave 14.7 g of crude product. The blue-fluorescing product was isolated by means of TLC. R_{f} (1:2 toluene:petroleum ether)=0.6.

### II. Preparation of Compounds I

### Example 1

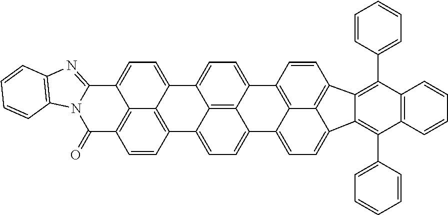

### Example 1.1

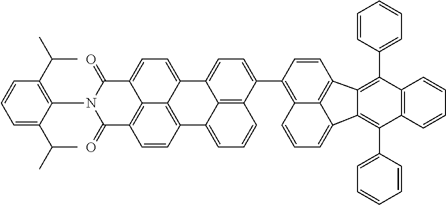

A mixture of 120 ml of toluene, 2.31 g (4.8 mmol) of the compound from example I.1 and 3.0 g (4.8 mmol) of the compound from example I.a was heated to 40° C.

Thereafter, a solution of 18.0 g of potassium carbonate, 120 ml of water and 48 ml of ethanol, and also 3.0 g (2.58 mmol) of tetrakistriphenylphosphinepalladium(0) were added, and the reaction mixture was stirred at 50° C. for 2 h. After removal of the solvent under reduced pressure and purification by chromatography on silica gel (10:1 toluene:petroleum ether), 2.8 g (67%) of the title compound were obtained as a magenta-red compound. R_{f} (10:1 toluene:petroleum ether)=0.72.

### Example 1.2

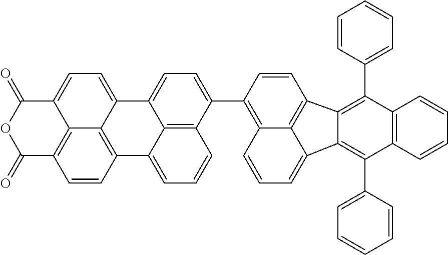

A mixture of 150 ml of 2-methyl-2-butanol, 2.8 g (3.2 mmol) of the compound from example 1.1 and 7.4 g (108 mmol) of KOH was stirred under reflux (102° C.) for 3 hours. After cooling, the reaction mixture was stirred with 900 ml of dichloromethane, 17 ml of glacial acetic acid and 30 ml of water for 5 hours. The organic phase was extracted by shaking with water and concentrated. This gave 2.7 g (quantitative yield) of the title compound as a magenta-red compound.

R_{f} (10:1 toluene:ethyl acetate)=0.73.

### Example 1.3

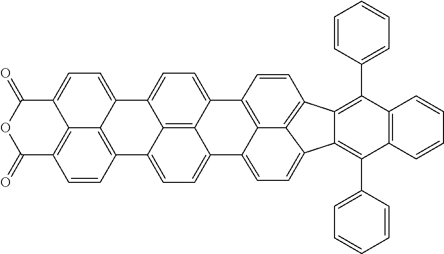

2.7 g (3.7 mmol) of the compound from example 1.2 and a solution of 8.0 g of iron(III) chloride and 100 ml of nitromethane were added to 200 ml of argon-saturated dichloromethane. The reaction mixture was stirred at room temperature overnight. After extraction with water, the organic phase was concentrated to 300 ml. The product which precipitated out was filtered off with suction, washed with dichloromethane and hot water, and dried. This gave 1.46 g of a blue compound (54% yield). R_{f} (dichloromethane)=0.0

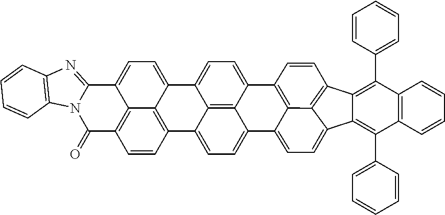

A mixture of 27 ml of quinoline, 1.4 g (2.0 mmol) of the compound from example 1.3, 0.26 g (2.36 mmol) of 1,2-diaminobenzene and 0.43 g (2.36 mmol) of zinc acetate was stirred at 140° C. for 3 hours. After cooling, 300 ml of 1 molar hydrochloric acid were added and the mixture was stirred for 1 hour. The product which precipitated out was filtered off with suction, washed with hot water and ethanol, and dried. The crude product was stirred with 50 ml of N-methylpyrrolidone (NMP) at 200° C. The mixture was allowed to cool gradually, 10 ml of toluene were added and the precipitate was filtered off with suction. The further purification was effected by stirring the crude product with 20 ml of quinoline at 215° C. overnight. The precipitate which formed in the course of gradual cooling was filtered off with suction at 100° C. and washed with dichloromethane, dilute hydrochloric acid and water. This gave 0.68 g (44%) of the title compound as a sparingly soluble blue solid. For further purification, 170 mg of the title compound were sublimed in a gradient sublimation apparatus. The temperatures in the individual zones were 500° C., 450° C. and 400° C. In the region of 400° C., 31 mg (18.2% based on the sublimation) of the product were obtained as a sublimate.

### Example 2

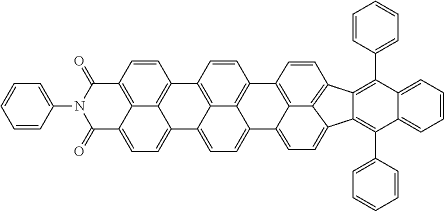

### Example 2.1

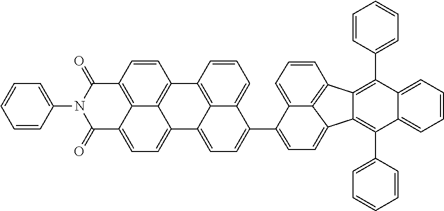

0.92 g (1.9 mmol) of the compound from example I.1 and 1.00 g (1.9 mmol) of the boron compound from example I.o were dissolved in 40 ml of toluene at 40° C.

Thereafter, a solution of 6.0 g of potassium carbonate, 40 ml of water and 16 ml of ethanol, and also 1.0 g (0.86 mmol) of tetrakistriphenylphosphinepalladium(0) were added, and the reaction mixture was stirred at 40° C. for 2 h. The reaction mixture was extracted by shaking with dichloromethane and water, and the organic phase was removed and concentrated. This gave 3.0 g of the title compound as the crude product.

### Example 2.2

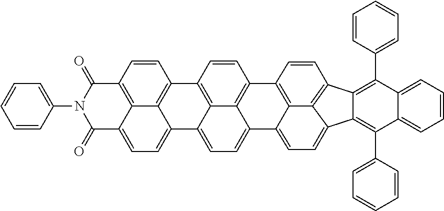

A mixture of 150 ml of argon-saturated chlorobenzene, 3.0 g of the compound from example 2.1 (crude product, approx. 50%) and 6.5 g of aluminum trichloride

(suspended in 50 ml of chlorobenzene) was stirred at 110° C. for 2.5 hours. The reaction mixture was admixed with dichloromethane and extracted with water; the organic phase was concentrated under reduced pressure. The crude product obtained was chromatographed using a suction filter with silica gel using 100:1 toluene:ethyl acetate as the eluent. This gave 100 mg of the title compound as a blue solid.

### Example 3

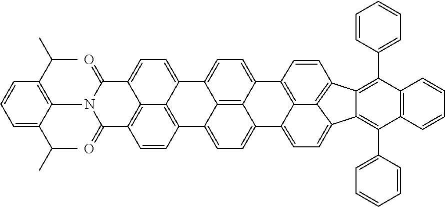

0.1 g (0.11 mmol) of the compound from example 1.1 and a solution of 0.32 g of iron(III) chloride and 10.0 ml of nitromethane were added to 20.0 ml of argon-saturated dichloromethane. The reaction mixture was stirred at −5° C. for a further 2 hours. After extraction by shaking with water, the organic phase was concentrated, dissolved in toluene and chromatographed with toluene using silica gel. This gave 74 mg of the title compound as a blue solid (74% yield). R_{f} (10:1 toluene/ethyl acetate)=0.63.

### III. Production of the Cells

### Materials:

C60: obtainable from CreaPhys,
Bphen: obtainable from Fluka,
Compound from example 1
MoO₃: obtainable from Sigma-Aldrich

### Substrate:

ITO was sputtered onto a glass substrate in a thickness of 120 nm. The specific resistivity was 15 Ωcm and the root-mean-square roughness (RMS) was less than 2 nm. Before the deposition of further layers, the substrate was treated with ozone under UV light for 15 minutes (UV-ozone cleaning).

Bilayer cells (cells of two-layer structure) and bulk heterojunction cells (BHJ cells) were produced under high vacuum (pressure approx. 2×10⁻⁶ mbar).

### Bilayer Cell

### (ITO/MoO₃/Inventive Compound of the Formula I/C₆₀/Bphen/Ag):

The bilayer cell was produced by successive vapor deposition of an inventive compound of the formula I and C60 onto MoO₃-coated ITO substrate. The vapor deposition rate for both layers was 0.3 nm/second. Thereafter, the Bphen layer was applied by vapor deposition of Bphen. Finally, an Ag layer was applied by vapor deposition as a top electrode. The vaporization temperatures are reported in table 1. The cell had an area of 0.04 cm². The cell structure of the bilayer cell is shown in table 2.

TABLE 1
Vaporization temperatures

| Compound | Vaporization temperature [° C.] |
|---|---|
| Bphen | 130 |
| C60 | 380 |
| Compound from example 1 | 450 |

TABLE 2
Cell structure of the bilayer cell

| | | Compound from | | |
|---|---|---|---|---|
| ITO | MoO₃ [nm] | example 1 [nm] | C60 [nm] | Bphen/Ag [nm] |
| | 5 | 10 | 35 | 5/100 |
| | | 20 | | |

### BHJ Cell:

### (ITO/MoO₃/(Inventive Compound of the Formula I:C60)/C60/Bphen/Ag)

To produce the BHJ cell (bulk heterojunction cell), an inventive compound of the formula I and C60 were covaporized. The weight ratio of inventive compound I to C60 was 1:1.5. The Bphen and Ag layers were applied by vapor deposition as described for the bilayer cell. The cell structure of the BHJ cell is reported in table 3.

TABLE 3
Cell structure of the BHJ cell

| | MoO₃ | Layer thickness: compound from | | Bphen/Ag |
|---|---|---|---|---|
| ITO | [nm] | example 1 + C₆₀ [nm] | C60 [nm] | [nm] |
| | 5 | 15 | 25 | 5/100 |
| | | 30 | | |
| | | 45 | | |
| | | 60 | | |

### Measurements:

The solar simulator used was an AM 1.5 Simulator from Solar Light Co. Inc. with a xenon lamp (model 16S-150 V3). The UV range below 380 nm was filtered and the current-voltages were measured under ambient conditions. The intensity of the solar simulator was calibrated with a monocrystalline FZ solar cell (Fraunhofer ISE) and the deviation factor was determined to be approximately 1.0.

### Results:

TABLE 4
Bilayer cell: ITO/MoO₃/compound from example 1/C60/Bphen/Ag)

| | Layer | | | | |
|---|---|---|---|---|---|
| | thickness | | FF | I_{SC} | V_{OC} |
| Compound | [nm] | η [%] | [%] | [mA/cm²] | [mV] |
| Example 1 | 10 | 0.8 | 44 | −3.1 | 580 |
| | 20 | 0.3 | 20 | −2.1 | 640 |

TABLE 5
BHJ cell: (ITO/MoO₃/(compound from example 1:C60)/C60/Bphen/Ag)

| | Layer | | | | |
|---|---|---|---|---|---|
| | thickness | | FF | I_{SC} | V_{OC} |
| Compound | [nm] | η [%] | [%] | [mA/cm²] | [mV] |
| Example 1 | 15 | 1.6 | 55 | −5.4 | 560 |
| | 30 | 1.6 | 39 | −7.3 | 560 |
| | 45 | 1.5 | 38 | −6.9 | 560 |
| | 60 | 1.1 | 35 | −5.7 | 560 |

1. A terrylene compound of the general formula (I) 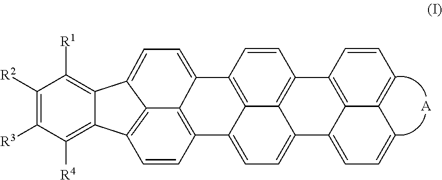
in which
R¹ and R⁴ are each independently selected from hydrogen and in each case unsubstituted or substituted alkyl, aryl, thiophenyl and oligothiophenyl;
R² and R³ are each independently selected from hydrogen and in each case unsubstituted or substituted alkyl, aryl, thiophenyl and oligothiophenyl;
where in each case at least two adjacent radicals selected from the R¹, R², R³ and R⁴ radicals, together with the carbon atoms of the benzene ring to which they are bonded, may also be a fused ring system having 1, 2, 3, 4, 5, 6, 7 or 8 further rings, and
A with the carbon atoms to which it is bonded is a fused monocyclic, dicyclic, tricyclic, tetracyclic, pentacyclic or hexacyclic ring system which optionally bears one or more independently selected substituents.

2. A terrylene compound according to claim 1, wherein R¹, R², R³ and R⁴ are each independently selected from hydrogen, unsubstituted alkyl, aralkyl, unsubstituted aryl, alkaryl, thiophenyl or oligothiophenyl, where the two latter radicals may bear one or more alkyl substituents.

3. A terrylene compound according to either of claims 1 and 2, wherein R¹ and R⁴ are each independently selected from hydrogen and groups of the general formulae (III.1) to (III.12) 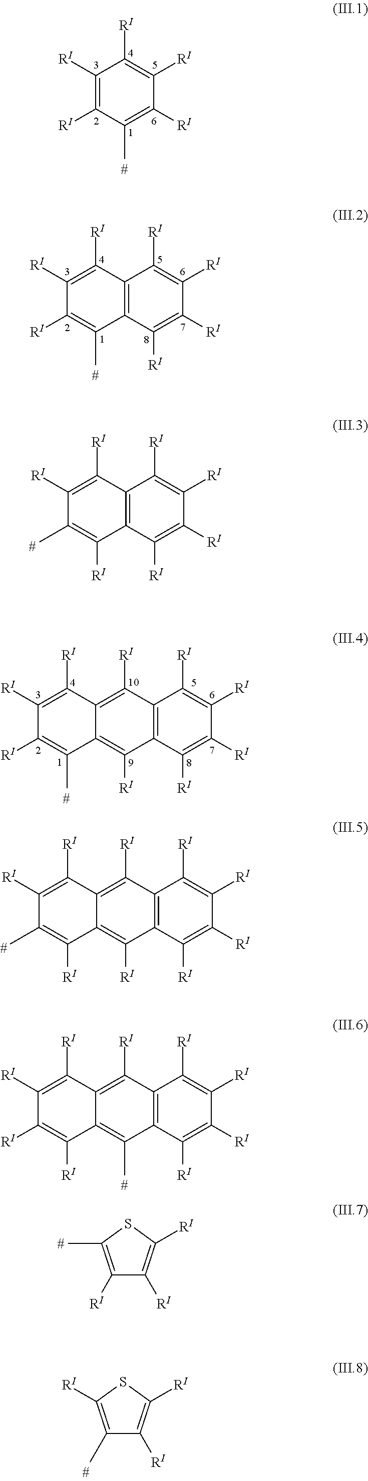 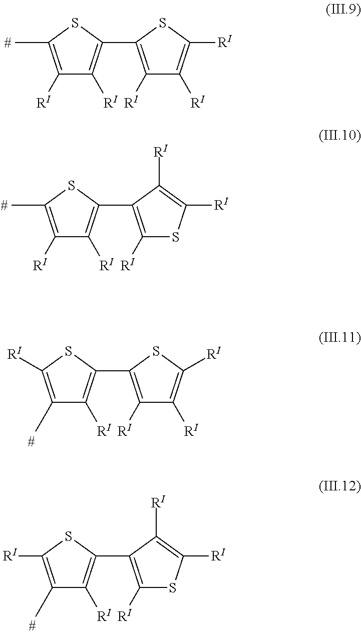
in which
# represents the bonding site to the benzene ring, and
R^{I} is in each case independently selected from hydrogen, unsubstituted alkyl, aryl, aralkyl and alkaryl.

4. A terrylene compound according to any of the preceding claims, wherein R¹ and R⁴ are each independently selected from hydrogen and groups of the general formulae (III.1a), (III.1b), (III.1c), (III.1d), (III.2a), (III.4a), (III.7a), (III.7b), (III.8a), (III.8b), (III.9a), (III.9b), (III.10a), (III.10b), (III.11a), (III.11b), (III.12a) and (III.12b) 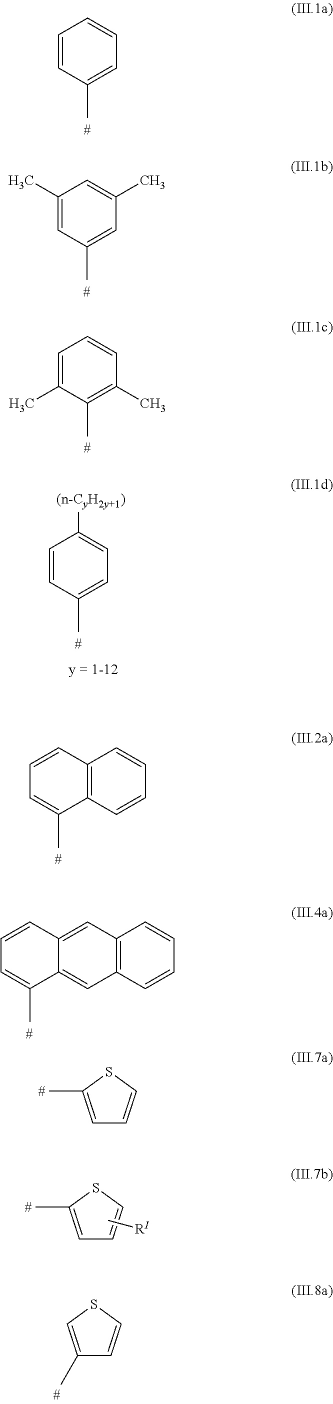 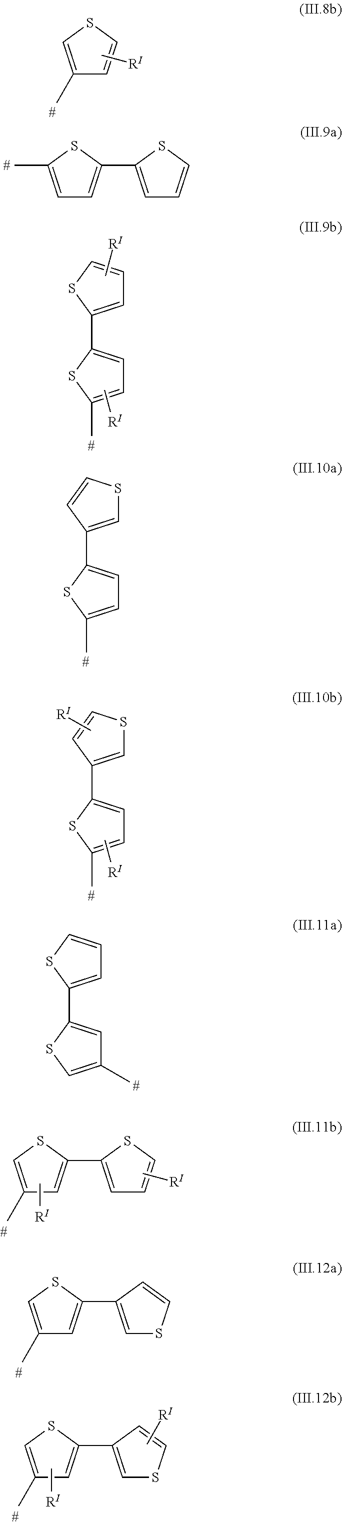
in which
# represents the bonding site to the benzene ring; and
R^{I} is hydrogen or C₁-C₈-alkyl.

5. A terrylene compound according to any of the preceding claims, wherein R¹ and R⁴ are each independently selected from phenyl, thiophen-2-yl and 5-methylthiophen-2-yl.

6. A terrylene compound according to any of claims 1 to 5, wherein the R² and R³ groups in the compounds of the general formula (I) are each independently selected from hydrogen and groups of the general formulae (IV.1) to (IV.12) 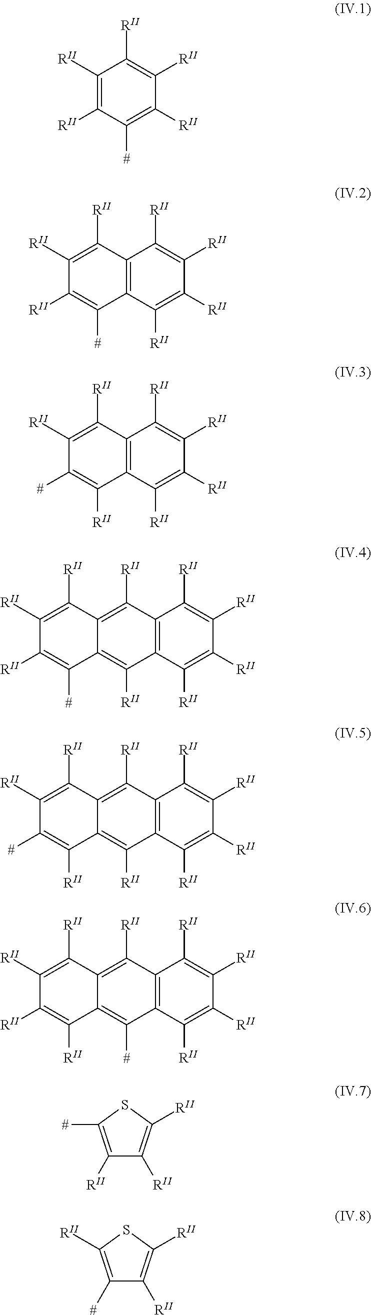 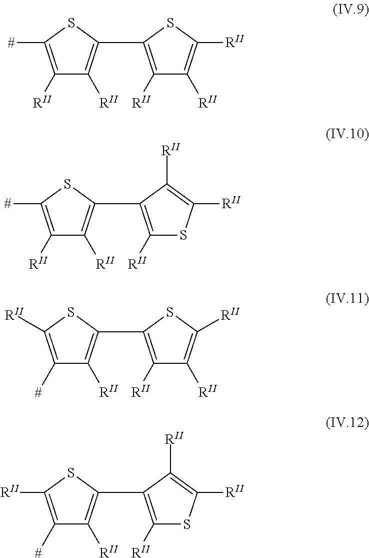
in which
# represents the bonding site to the benzene ring, and
R^{II} is in each case independently selected from hydrogen, unsubstituted alkyl, aryl, aralkyl and alkaryl.

7. A terrylene compound according to any of claims 1 to 6, wherein R² and R³ are each independently selected from hydrogen and groups of the general formulae (IV.1a), (IV.1b), (IV.1c), (IV.1d), (IV.2a), (IV.4a), (IV.7a), (IV.7b), (IV.8a), (IV.8b), (IV.9a), (IV.9b), (IV.10a), (IV.10b), (IV.11a), (IV.11b), (IV.12a) and (IV.12b): 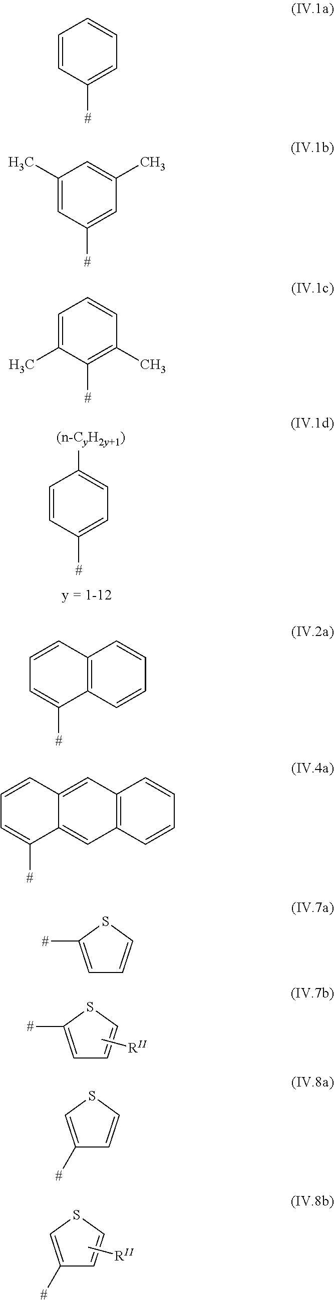 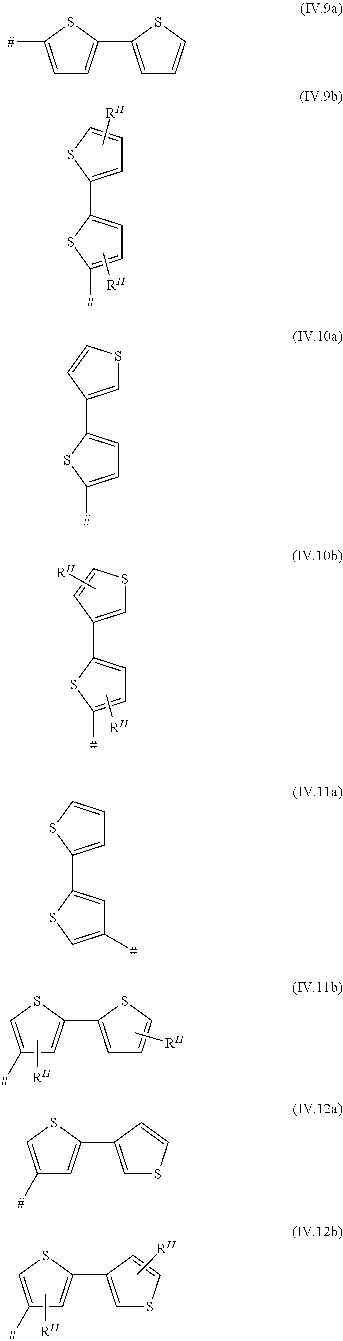
in which
# represents the bonding site to the benzene ring; and
R^{II} is hydrogen or C₁-C₈-alkyl.

8. A terrylene compound according to any of claims 1 to 7, wherein the R² and R³ groups are each independently selected from hydrogen and phenyl.

9. A terrylene compound according to any of claims 1 to 5, wherein the R² and R³ groups together are a group selected from groups of the general formulae (V.1), (V.2) and (V.3) 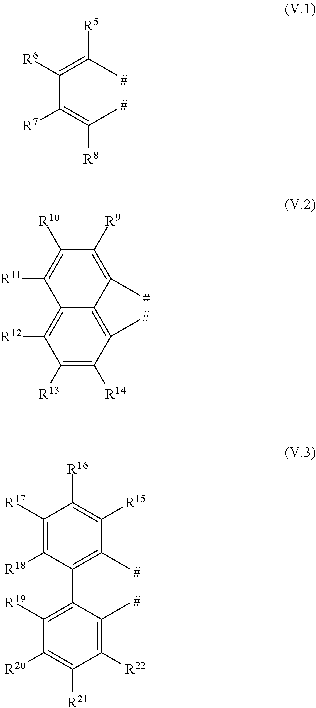
in which
# in each case represents a bonding site to the benzene ring;
R⁵, R⁶, R⁷ and R⁸ are each independently hydrogen, C₁-C₂₀-alkyl or phenyl, where phenyl may be unsubstituted or may bear 1, 2 or 3 R^{a} radicals;
R⁹, R¹⁰, R¹¹, R¹², R¹³ and R¹⁴ are each independently hydrogen, C₁-C₂₀-alkyl or phenyl, where phenyl may be unsubstituted or may bear 1, 2 or 3 R^{a} radicals; and
R¹⁵, R¹⁶, R¹⁷, R¹⁸, R¹⁹, R²⁰, R²¹ and R²² are each independently hydrogen, C₁-C₂₀-alkyl or phenyl, where phenyl may be unsubstituted or may bear 1, 2 or 3 R^{a} radicals, in which
R^{a} is C₁-C₁₀-alkyl.

10. A terrylene compound according to claim 9, wherein the R² and R³ groups together are a group of the formula V.1 where R⁵, R⁶, R⁷ and R⁸ are each hydrogen, or
together are a group of the formula V.2 where R⁹, R¹⁰, R¹¹, R¹², R¹³ and R¹⁴ are each hydrogen, or
together are a group of the formula V.3 where R¹⁵, R¹⁶, R¹⁷, R¹⁸, R¹⁹, R²⁰, R²¹ and R²² are each hydrogen.

11. A terrylene compound according to any of the preceding claims, in which A is selected from groups of the general formulae (VI.1), (VI.2), (VI.3), (VI.4), (VI.5) and (VI.6) 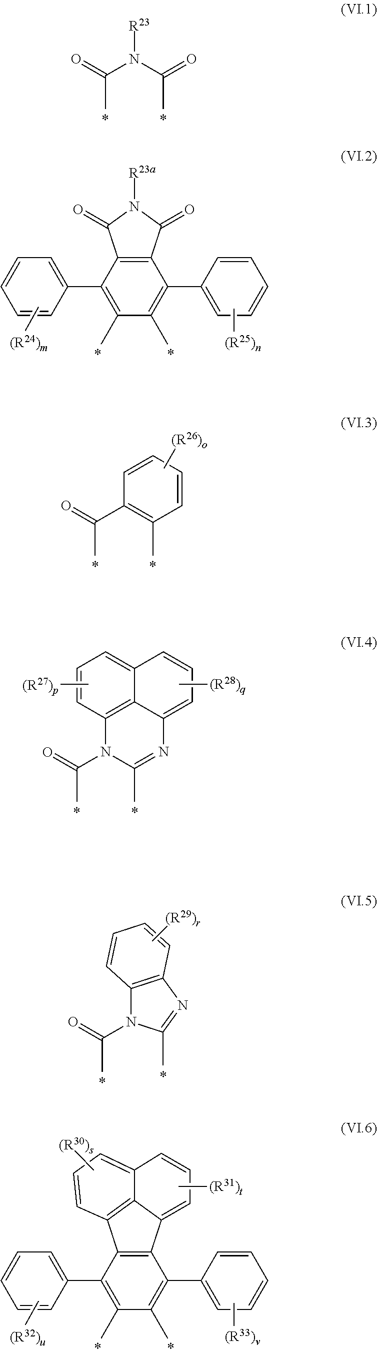
in which
in each case represents the bonding site to the terrylene base skeleton,
R²³ is hydrogen, alkyl, aryl, alkaryl or aralkyl,
R^{23a} is hydrogen, alkyl, aryl, alkaryl or aralkyl,
R²⁴, R²⁵ are each independently alkyl, aryl, alkaryl or aralkyl, where two R²⁴ and/or R²⁵ radicals bonded to adjacent carbon atoms may also be a fused-on benzene ring,
R²⁶ is alkyl, aryl, alkaryl or aralkyl, where two R²⁶ radicals bonded to adjacent carbon atoms may be a fused-on benzene ring,
R²⁷, R²⁸ are each independently alkyl, aryl, alkaryl or aralkyl, where two R²⁷ and/or R²⁸ radicals bonded to adjacent carbon atoms may also be a fused-on benzene ring,
R²⁹ is alkyl, aryl, alkaryl or aralkyl, where two R²⁹ radicals bonded to adjacent carbon atoms may also be a fused-on benzene ring,
R³⁰, R³¹ are each independently cyano, alkyl, aryl, alkaryl or aralkyl,
R³², R³³ are each independently cyano, alkyl, aryl, alkaryl or aralkyl,
m, n, u, v are each independently 0, 1, 2 or 3, and
o, p, q, r, s, t are each independently 0, 1 or 2.

12. A terrylene compound according to claim 11, in which, in the groups of the general formulae (VI.1), (VI.2), (VI.3), (VI.4), (VI.5) and (VI.6),
in each case represents the bonding site to the terrylene base skeleton,
R²³, R^{23a} are each hydrogen, C₁-C₂₀-alkyl, C₆-C₁₄-aryl-C₁-C₂₀-alkyl, C₆-C₁₄-aryl or C₁-C₂₀-alkyl-C₆-C₁₄-aryl,
R²⁴, R²⁵ are each independently C₁-C₂₀-alkyl or phenyl, where phenyl may be unsubstituted or may bear 1, 2 or 3 R^{b} radicals, and where two R²⁴ and/or R²⁵ radicals bonded to adjacent carbon atoms may also be a fused-on benzene ring,
R²⁶ is C₁-C₂₀-alkyl or phenyl, where phenyl may be unsubstituted or may bear 1, 2 or 3 R^{b} radicals, and where two R²⁶ radicals bonded to adjacent carbon atoms may be a fused-on benzene ring,
R²⁷, R²⁸ are each independently C₁-C₂₀-alkyl or phenyl, where phenyl may be unsubstituted or may bear 1, 2 or 3 R^{b} radicals, and where two R²⁷ and/or R²⁸ radicals bonded to adjacent carbon atoms may also be a fused-on benzene ring,
R²⁹ is C₁-C₂₀-alkyl or phenyl, where phenyl may be unsubstituted or may bear 1, 2 or 3 R^{b} radicals, and where two R²⁹ radicals bonded to adjacent carbon atoms may also be a fused-on benzene ring,
R³⁰, R³¹ are each independently C₁-C₂₀-alkyl, cyano or phenyl, where phenyl may be unsubstituted or may bear 1, 2 or 3 R^{b} radicals,
R³², R³³ are each independently C₁-C₂₀-alkyl, cyano or phenyl, where phenyl may be unsubstituted or may bear 1, 2 or 3 R^{b} radicals,
m, n, u, v are each independently 0, 1, 2 or 3,
o, p, q, r, s, t are each 0, 1 or 2,
R^{b} is C₁-C₁₀-alkyl, where different R^{b} radicals may each have identical or different definitions.

13. An organic solar cell with a photoactive region which comprises at least one organic donor material in contact with at least one organic acceptor material, said donor material and said acceptor material forming a donor-acceptor heterojunction, and said photoactive region comprising at least one terrylene compound of the formula (I) as defined in any of claims 1 to 12.

14. The organic solar cell according to claim 13, wherein the terrylene compound of the formula (I) as defined in any of claims 1 to 11 is used as an electron donor material.

15. The organic solar cell according to either of claims 13 and 14, wherein the photoactive region comprises at least one terrylene compound of the formula (I) in combination with at least one further different semiconductor material.

16. The organic solar cell according to claim 15, wherein the photoactive region comprises, as a further semiconductor material, at least one fullerene and/or fullerene derivative.

17. The organic solar cell according to claim 16, wherein the photoactive region comprises, as a further semiconductor material, C60 or [6,6]-phenyl-C61-butyric acid methyl ester.

18. The organic solar cell according to any of claims 13 to 17 comprising photoactive donor-acceptor junctions in the form of a bulk heterojunction.

19. The organic solar cell according to any of claims 13 to 18 in the form of a single cell or in the form of a tandem cell or in the form of a multijunction cell.

20. The organic solar cell according to any of claims 13 to 19 in the form of a tandem cell, in which one subcell comprises a terrylene compound of the general formula (I) and at least one fullerene or fullerene derivative.

21. The tandem cell according to claim 20, which additionally comprises at least one subcell whose absorption maximum in the relatively short-wave spectral region is from 400 to 650 nm.

22. A process for preparing a terrylene compound of the general formula (I.a) 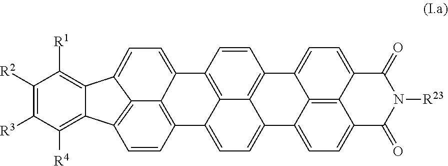
in which
R¹, R², R³, R⁴ and R²³ are each as defined in any of claims 1 to 12, by
i) reacting a compound of the formula VII 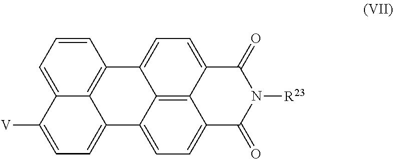
in a coupling reaction with a compound of the formula VIII 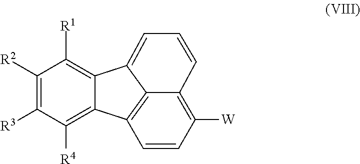
in which
R¹, R², R³ and R⁴ are each as defined above,
one of the V and W radicals is chlorine, bromine or iodine and the other V or W radical is a B(OR′)(OR″) radical in which
R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene which may be substituted by 1, 2, 3, 4, 5, 6, 7 or 8 substituents selected from C₁-C₄-alkyl, C₅-C₈-cycloalkyl, C₆-C₁₄-aryl or hetaryl,
to obtain at least one compound of the formula (IX) 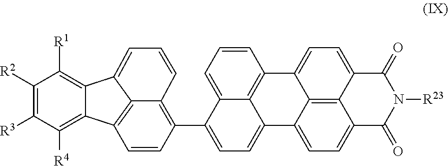
ii) subjecting the reaction product (IX) obtained in step (i) to a cyclodehydrogenation to obtain at least one terrylene compound of the general formula (I).

23. A process for preparing a terrylene compound of the general formula (I.b) 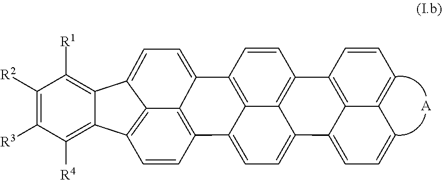
in which
R¹, R², R³ and R⁴ are each as defined in any of claims 1 to 12, and
A is selected from groups of the general formulae (VI.4) and (VI.5)
in which 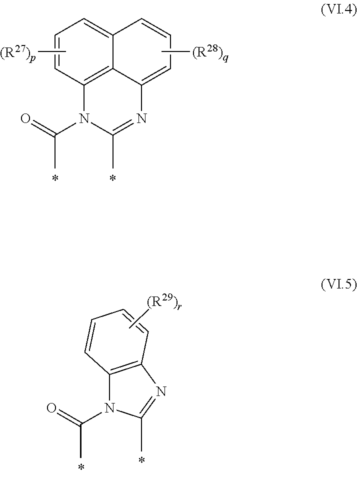
in each case represents the bonding site to the terrylene base skeleton,
R²⁷, R²⁸ are each independently unsubstituted alkyl, aryl, aralkyl or alkaryl, where two R²⁷ and/or R²⁸ radicals bonded to adjacent carbon atoms may also be a fused-on benzene ring,
R²⁹ is unsubstituted alkyl, aryl, alkaryl or aralkyl, where two R²⁹ radicals bonded to adjacent carbon atoms may also be a fused-on benzene ring,
p, q, r are each independently 0, 1 or 2,
by
i) reacting a compound of the formula VII.a 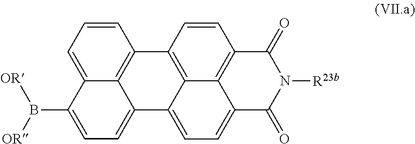
in which
R^{23b} is phenyl which is unsubstituted or substituted by 1 or 2 C₁-C₂₀-alkyl radicals,
R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene optionally substituted by 1, 2, 3, 4, 5, 6, 7 or 8 substituents selected from C₁-C₄-alkyl, C₅-C₈-cycloalkyl, C₆-C₁₄-aryl and hetaryl,
in a coupling reaction with a compound of the formula VIII 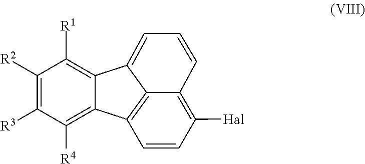
in which
R¹, R², R³ and R⁴ are each as defined above, and
Hal is chlorine, bromine or iodine,
to obtain at least one compound of the formula (IX.a) 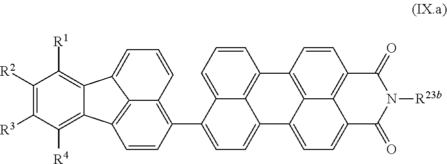
ii) subjecting the reaction product (IX.a) obtained in step (i) to a hydrolysis to obtain at least one anhydride (X) 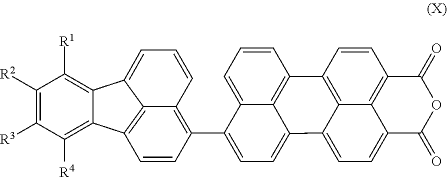
iii) subjecting the anhydride (X) obtained in step (ii) to a cyclodehydrogenation to obtain at least one compound (XI) 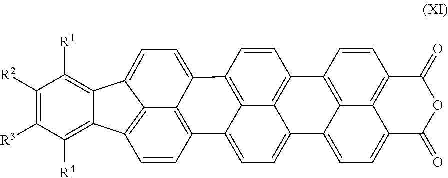
iv) subjecting the reaction product from step iii) to an imidation with an amine of the formula (XII.a) or (XII.b) 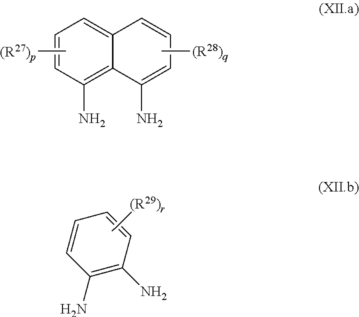
in which
in each case represents the bonding site to the terrylene base skeleton,
R²⁷, R²⁸, R²⁹, p, q and r are each as defined above,
to obtain at least one terrylene compound of the general formula (I.b).

24. A process for preparing a terrylene compound of the general formula (I) 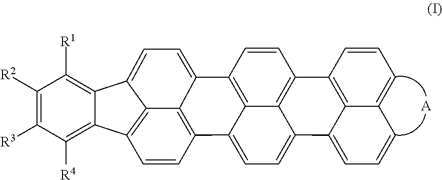
in which
R¹, R², R³, R⁴ and A are each as defined in any of claims 1 to 11,
by
i) reacting a compound of the formula (XIII) 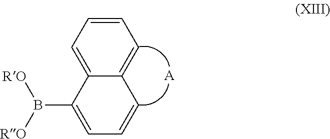
in which
A is as defined above,
R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene optionally substituted by 1, 2, 3, 4, 5, 6, 7 or 8 substituents selected from C₁-C₄-alkyl, C₅-C₈-cycloalkyl, C₆-C₁₄-aryl or hetaryl,
in a coupling reaction with a compound of the formula (XIV.a) or (XIV.b) 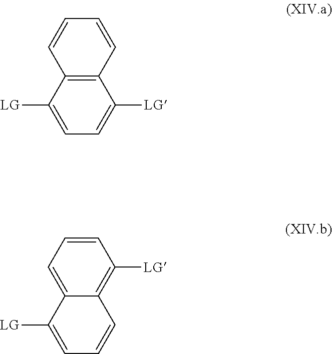
in which
LG is tosylate, chlorine, bromine or iodine,
LG′ is tosylate, chlorine, bromine or iodine,
to obtain at least one compound of the formula (XV) 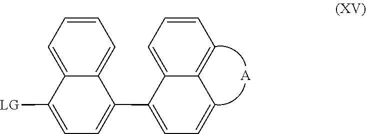
ii) subjecting the reaction product obtained in step (i) to a cyclodehydrogenation to obtain at least one compound of the general formula (XVI) 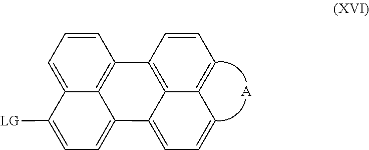
iii) reacting the reaction product (XVI) obtained in step (ii) in a coupling reaction with a compound of the formula (XVII) 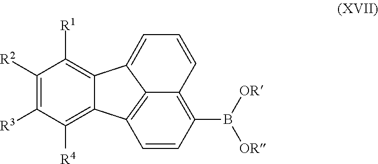
in which
R¹, R², R³ and R⁴ are each as defined above,
R′ and R″ are each independently hydrogen, C₁-C₃₀-alkyl, C₅-C₈-cycloalkyl, C₆-C₁₄-aryl or hetaryl, or R′ and R″ together are C₂-C₄-alkylene optionally substituted by 1, 2, 3, 4, 5, 6, 7 or 8 substituents selected from C₁-C₄-alkyl, C₅-C₈-cycloalkyl, C₆-C₁₄-aryl or hetaryl,
to obtain at least one compound of the general formula (XVIII) 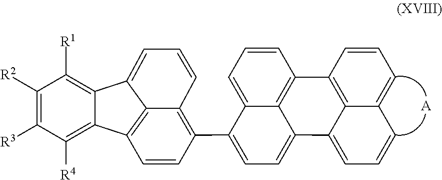
iv) subjecting the reaction product (XVIII) obtained in step (iii) to a cyclodehydrogenation to obtain at least one compound of the general formula (I).

25. The use of at least one terrylene compound of the formula (I) as defined in any of claims 1 to 12 as an electron donor material in organic solar cells.
